(19) United States
(12) Patent Application Publication
(10) Pub. No.: US 2022/0370556 A1
(43) Pub. Date: Nov. 24, 2022

(54) Method of Treatment

(71) Applicant: Volution Immuno Pharmaceuticals SA, 1211 Geneve 3 (CH)

(72) Inventors: Wynne H. Weston-Davies, Cheltenham (GB); Miles Nunn, 3 Geneve (CH)

(21) Appl. No.: 17/761,753

(22) PCT Filed: Sep. 27, 2019
(86) PCT No.: PCT/EP2019/076313

§ 371 Date: Mar. 18, 2022

(51) Int. Cl. A61K 38/17; A61P 7/00; A61K 45/06

(52) U.S. Cl. CPC A61K 38/1767; A61P 7/00; A61K 45/06

(57) ABSTRACT

The present invention relates to methods of treating or preventing hematopoietic stem cell transplant-associated thrombotic microangiopathy (HSCT-TMA) in a subject, which comprises administering to the subject a therapeutically or prophylactically effective amount of an agent which is a protein comprising amino acids 19 to 168 of the amino acid sequence in FIG. 2 (SEQ ID NO: 2) or a functional equivalent of this protein. Said protein of FIG. 2 of the present application has been designated in the prior art as Coversin, Nomacopan, EV576 or OmCI protein. Alternatively the agent is a nucleic acid molecule encoding a protein comprising amino acids 19 to 168 of the amino acid sequence in FIG. 2 (SEQ ID NO: 2) or a functional equivalent of this protein.

The present invention relates to methods of treating and preventing hematopoietic stem cell transplant-associated thrombotic microangiopathy (HSCT-TMA).

All documents mentioned in the text and listed at the end of this description are incorporated herein by reference.

### BACKGROUND TO THE INVENTION

### Complement

The complement system is an essential part of the body's natural defence mechanism against foreign invasion and is also involved in the inflammatory process. More than 30 proteins in serum and at the cell surface are involved in the functioning and regulation of the complement system. Recently, it has become apparent that, as well as the approximately 35 known components of the complement system, which may be associated with both beneficial and pathological processes, the complement system itself interacts with at least 85 biological pathways with functions as diverse as angiogenesis, platelet activation and haemostasis, glucose metabolism and spermatogenesis.

The complement system is activated by the presence of materials that are recognised by the immune system as non-self. Three activation pathways exist: (1) the classical pathway which is activated by IgM and IgG complexes or by recognition of carbohydrates; (2) the alternative pathway which is activated by non-self surfaces (lacking specific regulatory molecules) and by bacterial endotoxins; and (3) the lectin pathway which is activated by binding of mannan-binding lectin (MBL) to mannose residues on the surface of a pathogen. The three pathways comprise parallel cascades of events that result in the production of complement activation through the formation of similar C3¹ and C5 convertases on cell surfaces, resulting in the release of acute mediators of inflammation (C3a and C5a) and the formation of the membrane ¹ It is conventional to refer to the components of the complement pathway by the letter “C” followed by a number, such as “3”, such that “C3” refers to complement protein C3. Some of these components are cleaved during activation of the complement system and the cleavage products are given lower case letters after the number. Thus, C5 is cleaved into fragments which are conventionally labelled C5a and C5b. The complement proteins do not necessarily act in their number order and so the number does not necessarily give any indication of the order of action. This naming convention is used in this application. attack complex (MAC). The parallel cascades involved in the classical (here defined as classical via C1q and lectin via MBL) and alternative pathways are shown in FIG. 1. The classical complement pathway, the alternative complement pathway and the lectin complement pathway are herein collectively referred to as the complement pathways. C5b initiates the ‘late’ or ‘terminal’ events of complement activation. These comprise a sequence of polymerization reactions in which the terminal complement components interact to form the MAC, which creates a pore in the cell membranes of some pathogens which can lead to their death or activates the body's own cells without causing lysis. The terminal complement components include C5b (which initiates assembly of the membrane attack system), C6, C7, C8 and C9.

### LTB4

Leukotriene B4 (LTB4) is the most powerful chemotactic and chemokinetic eicosanoid described and promotes adhesion of neutrophils to the vascular endothelium via upregulation of integrins [1]. It is also a complete secretagogue for neutrophils, induces their aggregation and increases microvascular permeability. LTB4 recruits and activates natural killer cells, monocytes and eosinophils. It increases superoxide radical formation [2] and modulates gene expression including production of a number of proinflammatory cytokines and mediators which may augment and prolong tissue inflammation [3,4]. LTB4 also has roles in the induction and management of adaptive immune responses. For example, regulation of dendritic cell trafficking to draining lymph nodes [5,6], Th2 cytokine IL-13 production from lung T cells [7], recruitment of antigen-specific effector CD8+T cells [8] and activation and proliferation of human B lymphocytes [9].

LTB4 and the hydroxyeicosanoids mediate their effects through the BLT1 and BLT2 G-protein coupled receptors [10,11]. Human BLT1 is a high affinity receptor (Kd 0.39-1.5 nM; [12]) specific for LTB4 with only 20-hydroxy LTB4 and 12-epi LTB4 able to displace LTB4 in competitive binding studies [13]. Human BLT2 has a 20-fold lower affinity (Kd 23 nM) for LTB4 than BLT1 and is activated by binding a broader range of eicosanoids including 12-epi LTB4, 20-hydroxy LTB4, 12(S)- and 15(S)-HETE and 12(S)- and 15(S)-HPETE [13]. Human BLT2 has 45.2 and 44.6% amino acid identity with human and mouse BLT1, while human and mouse BLT2 have 92.7% identity [11].

Human BLT1 is mainly expressed on the surface of leukocytes, though it has recently been described in endothelial cells and vascular smooth muscle cells. Human BLT2 is expressed in a broader range of tissue and cell types. A number of specific antagonists of BLT1 and BLT2 have been described which inhibit activation, extravasation and apoptosis of human neutrophils [14].

A number of marketed drugs target the eicosanoids. These include the glucocorticoids which modulate phospholipase A2 (PLA2) and thereby inhibit release of the eicosanoid precursor arachidonic acid (AA) [15]. Non-steroidal anti-inflammatory drugs (NSAID) and other COX2 inhibitors which prevent synthesis of the prostaglandins and thromboxanes [16]. There are also a number of leukotriene (LK) modifiers which either inhibit the 5-LOX enzyme required for LTB4 synthesis and other leukotrienes (Zileuton; [17]), or that antagonise the CysLT1 receptor that mediates the effects of cysteinyl leukotrienes (Zafirlukast and Montelukast) [18]. The LK modifiers are orally available and have been approved by the FDA for use in the treatment of e.g. asthma. No drug that acts specifically on LTB4 or its receptors has yet reached the market.

### Hematopoietic Stem Cell Transplants

Hematopoietic stem cell transplantation (HSCT) involves the intravenous infusion of autologous or allogeneic stem cells to re-establish hematopoietic function in patients whose bone marrow or immune system is damaged or defective. More than 50,000 HSCTs are carried out annually worldwide and this number is increasing each year. Stem cell transplantation remains the last hope for patients with many different types of advanced or refractory diseases.

The HSCT procedure is often performed as part of therapy to eliminate a bone marrow infiltrative process, such as leukemia, or to correct congenital immunodeficiency disorders. HSCT can also be used to allow patients with cancer to receive higher doses of chemotherapy than bone marrow can usually tolerate—the bone marrow function is then salvaged by replacing the marrow with previously harvested stem cells. HSCT is used as a general term covering transplantation of blood progenitor/stem cells from any source (such as, bone marrow, peripheral blood or cord blood) to a subject, either the same subject as the stem cells were originally derived (an autologous HSCT), or to a different subject (an allogeneic transplant).

While transplantation-related mortality and morbidity rates have considerably decreased over recent years, there is still a relatively high death rate which results from HSCT-associated complications, such as thrombotic microangiopathy (TMA).

### Hematopoietic Stem Cell Transplant-Associated Thrombotic Microangiopathy (HSCT-TMA)

TMA is a small vascular occlusive disorder and the pathophysiology of TMA involves arteriolar and capillary platelet-mediated thromboses, associated ischemic tissue damage and fragmented red blood cells due to the shear stress across partially obstructed vessels. TMA occurs in 20-30% of HSCT recipients, and usually occurs within 100 days post-transplant [19]. The consequence of hematopoietic stem cell transplant-associated thrombotic microangiopathy (HSCT-TMA) is severe, with a mortality rates reported as high as 90-100%. Despite these high risks, for many patients, HSCT is often the only remaining therapeutic intervention with a curative goal.

While HSCT-TMA is more common after allogeneic HSCT, it is also remains a significant complication of autologous transplantation. HSCT-TMA usually presents as anemia, thrombocytopenia, renal impairments and pulmonary hypertension, and may involve gastrointestinal symptoms and central nervous system injury [20].

There is currently no accepted standard therapy for HSCT-TMA. New and effective treatments are therefore much needed in order to reduce the high mortality rate associated with HSCT-TMA.

### Complement Inhibitors

WO 2004/106369 (Evolutec Limited [21]) relates to complement inhibitors. A particular subset of the disclosed complement inhibitors are directed at C5 and prevent C5 being cleaved into C5a and C5b by any of the complement activation pathways. A particular example of such an inhibitor of C5 cleavage is a protein produced by ticks of the species Ornithdoros moubata, which in mature form is a protein consisting of amino acids 19 to 168 of the amino acid sequence shown in FIG. 4 of WO 2004/106369. In WO 2004/106369, this protein is known by the names “EV576”,“OmCI protein”, and “Coversin” and has more recently been known as “nomacopan” [22]. This protein is referred to herein as “nomacopan”.

In the tick, nomacopan is expressed as a pre-protein having a leader sequence comprising amino acids 1 to 18 of the amino acid sequence shown in FIG. 4 of WO 2004/106369 at the N-terminal end of the mature nomacopan protein. The leader sequence is cleaved off after expression. The mature protein has the sequence consisting of amino acids 19 to 168 of the amino acid sequence shown in FIG. 4 of WO 2004/106369 and FIG. 2 of the present application.

Nomacopan also has the ability to inhibit leukotriene B4 (LTB4) activity. The ability to bind LTB4 may be demonstrated by standard in vitro assays known in the art, for example by means of a competitive ELISA between nomacopan and an anti-LTB4 antibody competing for binding to labelled LTB4, by isothermal titration calorimetry or by fluorescence titration. There are a number of further patent applications, such as WO 2007/028968, WO 2008/029167, WO 2008/029169, WO 2011/083317, WO 2016/198133, WO 2017/0140903, WO 2018/0193120, WO 2018/0193121 and WO 2018/193122, which relate to the use of nomacopan or functional equivalents thereof in various applications. There is no experimental evidence in these applications that confirms the efficacy of nomacopan or any functional equivalent thereof in the treatment of HSCT-TMA.

In work leading to the present invention, the molecule nomacopan which binds LTB4 and which also inhibits the complement pathway by binding to C5, as discussed above, has been shown to ameliorate symptoms in HSCT-TMA patients. Nomacopan has the ability to inhibit both Complement (by inhibiting C5) and also LTB4 and is therefore particularly advantageous in the prevention and treatment of HSCT-TMA, either alone or in combination with other treatments.

### SUMMARY OF THE INVENTION

Nomacopan has been shown to ameliorate symptoms of TMA in HSCT-TMA patients. Nomacopan has the ability to inhibit Complement (by inhibiting C5) and is therefore particularly advantageous in the prevention and treatment of HSCT-TMA, either alone or in combination with other treatments. In the Example and FIG. 3, the use of nomacopan is demonstrated to resolve eight different markers of TMA, including hemolytic anemia, red blood cell fragment count, thrombocytopenia, increased lactate dehydrogenase (LDH) levels, proteinuria and/or increased creatinine, hypertension, neurological symptoms and gastrointestinal (GI) bleeds. Furthermore, the Example shows that nomacopan was able to significantly reduce complement activity over a prolonged period of time in HSCT-TMA patients.

The present inventors have therefore demonstrated that administration of the tick protein nomacopan (also referred to as EV576 and OmCI in the art and herein [21]) can be used to treat or prevent HSCT-TMA.

The invention therefore provides a method of treating or preventing HSCT-TMA, which comprises administering a therapeutically or prophylactically effective amount of an agent which is a protein comprising amino acids 19 to 168 of the amino acid sequence in FIG. 2 (SEQ ID NO: 2) or a functional equivalent of this protein.

The invention also provides an agent which is a protein comprising amino acids 19 to 168 of the amino acid sequence in FIG. 2 (SEQ ID NO: 2) or a functional equivalent of this protein for use in a method of treating or preventing HSCT-TMA.

The invention also provides a method of treating or preventing HSCT-TMA, comprising administering a therapeutically or prophylactically effective amount of an agent which is a nucleic acid molecule encoding a protein comprising amino acids 19 to 168 of the amino acid sequence in FIG. 2 (SEQ ID NO: 2) or a functional equivalent of this protein.

The invention also provides an agent which is a nucleic acid molecule encoding a protein comprising amino acids 19 to 168 of the amino acid sequence in FIG. 2 (SEQ ID NO: 2) or a functional equivalent of this protein for use in a method of treating or preventing HSCT-TMA.

The invention also provides a method of treating or preventing a HSCT-TMA, which comprises administering (a) a therapeutically or prophylactically effective amount of an agent which is a protein comprising amino acids 19 to 168 of the amino acid sequence in FIG. 2 (SEQ ID NO: 2) or a functional equivalent of this protein and (b) a second HSCT-TMA treatment.

The invention also provides (a) an agent which is a protein comprising amino acids 19 to 168 of the amino acid sequence in FIG. 2 (SEQ ID NO: 2) or a functional equivalent of this protein and (b) a second HSCT-TMA treatment, for use in a method of treating or preventing HSCT-TMA.

The invention also provides a method of treating or preventing HSCT-TMA, comprising administering (a) a therapeutically or prophylactically effective amount of an agent which is a nucleic acid molecule encoding a protein comprising amino acids 19 to 168 of the amino acid sequence in FIG. 2 (SEQ ID NO: 2) or a functional equivalent of this protein and (b) a second HSCT-TMA treatment.

The invention also provides (a) an agent which is a nucleic acid molecule encoding a protein comprising amino acids 19 to 168 of the amino acid sequence in FIG. 2 (SEQ ID NO: 2) or a functional equivalent of this protein and (b) a second HSCT-TMA treatment for use in a method of treating or preventing HSCT-TMA.

The invention also provides a method of reducing the amount of a second HSCT-TMA treatment that is required to treat or prevent HSCT-TMA, or reducing the duration of treatment with a second HSCT-TMA treatment that is required to treat or prevent HSCT-TMA, said method comprising administering a therapeutically or prophylactically effective amount of an agent which is a protein comprising amino acids 19 to 168 of the amino acid sequence in FIG. 2 (SEQ ID NO: 2) or a functional equivalent of this protein, or a nucleic acid molecule encoding said agent, and said second HSCT-TMA treatment.

### DETAILED DESCRIPTION

### HSCT-TMA and its Diagnosis

TMA is reported to occur in 10-30% of subjects which have undergone HSCT, and usually occurs within 100 days post-transplant [23]. HSCT-TMA is a TMA that occurs after the subject has undergone a HSCT. In general, the HSCT-TMA occurs within 100 days of the HSCT, but the HSCT-TMA can also occur within 200 days, within 150 days, within 125 days, within 80 days, or within 50 days of the HSCT. HSCT-TMA is also known as transplantation-associated thrombotic microangiopathy (TA-TMA) and HSCT-associated TMA. The presence of HSCT-TMA may be determined by routine diagnosis that is well understood in the art.

For example, diagnostic criteria for HSCT-TMA has been previously described in Cho et. al. [24]. In this example, diagnostic criteria for HSCT-TMA includes: (i) normal coagulation assays; (ii) schistocytosis, with ≥2 schistocytes per high powered field (≥2 HPF); (iii) increased serum lactose dehydrogenase (LDH); (iv) a negative Coomb's test; (v) thrombocytopenia, with a platelet count of <50,000/μL or a ≥50% reduction from previous counts; (vi) a decrease in hemoglobin concentration; and (vii) a decrease in serum haptoglobin.

An alternative diagnostic criteria for HSCT-TMA is Iacopino's criteria [25]. Here, the minimum criteria for HSCT-TMA was (1) simultaneous occurrence of at least two of the following parameters: microangiopathic hemolysis, thrombocytopenia, renal dysfunction, neurologic dysfunction, fever; (2) microangiopathic changes on blood smear with increased serum LDH activity. Clinical parameters for these criteria were defined as the following: microangiopathic hemolysis as intravascular hemolysis with a negative Coombs test; thrombocytopenia as a platelet count <150×10⁹/L; TMA-related fever as an unexplained oral temperature of greater than 38° C.; neurologic dysfunction defined as any abnormality observed during at least one neuropsychiatric examination. Renal dysfunction was diagnosed when either serum creatinine was >1.5 mg/dl or when its previously established elevated baseline value increased by 50%.

### Hematopoietic Stem Cell Transplantation

The subject may have previously had an autologous HSCT or an allogeneic HSCT. For example, the subject may have had an autologous HSCT to treat, or to try and treat, multiple myeloma, non-Hodgkin lymphoma, Hodgkin lymphoma, acute myeloid leukemia, neuroblastoma, germ cell tumours, autoimmune disorders (such as systemic lupus erythematosus, systemic sclerosis) and/or amyloidosis.

Alternatively, the subject may have had an allogeneic HSCT to treat acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin lymphoma, Hodgkin lymphoma, aplastic anemia, pure red-cell aplasia, paroxysmal nocturnal hemoglobinuria, Fanconi anemia, thalassemia major, sickle cell anemia, severe combined immunodeficiency, Wiskott-Aldrich syndrome, hemophagocytic lymphohistiocytosis, inborn errors of metabolism, epidermolysis bullosa, severe congenital neutropenia, Shwachman-Diamond syndrome, Diamond-Blackfan anemia, and/or leukocyte adhesion deficiency.

### Conditioning Prior to HSCT

Prior to a HSCT, it is typical for the subject to undergo a preparative or conditioning regimen. Such regimens function to provide immunosuppression sufficient to prevent rejection of the transplanted graft and/or to eradicate the disease for which the transplantation is being performed. The subject may have previously undergone a myeloablative or a non-myelo ablative conditioning regimen.

Myeloablative regimens are designed to kill all residual cancer cells in autologous or allogeneic transplantation and to cause immunosuppression for engraftment in allogeneic transplantation. Myeloablative regimens can be radiation-containing or non-radiation-containing regimens. Examples of radiation-containing a myeloablative regimens include: (i) total-body irradiation and cyclophosphamide; (ii) total-body irradiation and etoposide; (iii) total-body irradiation, etoposide and cyclophosphamide; and (iv) total-body irradiation and melphalan. Examples of non-radiation-containing myeloablative regimens include (i) cyclophosphamide and busulfan; (ii) busulfan and etoposide; (iii) cyclophosphamide, carmustine, and etoposide; (iv) cyclophosphamide, carmustine, etoposide, and cisplatin; and (v) carmustine, etoposide, cytarabine, and melphalan.

Non-myeloablative regimens are typically used in instances where an important contributing factor to effective treatment is a graft-versus-tumor effect mediated by the donor cells, for example in subjects with leukemia. In such cases, a toxic myeloablative preparative regimen is not necessarily required. Non-myeloablative regimens can result in a mixed chimerism which is the concurrent presence of donor and recipient hematopoietic cells in the subject. Non-myeloablative regimens use doses of chemotherapeutic drugs and radiation that are substantially lower than those of myeloablative regimens. Such regimens are usually beneficial for slow-growing tumors, such as those of chronic lymphocytic leukemia or chronic myeloid leukemia.

### Risk Factors for HSCT-TMA

The subject may have, be suspected of having, or may be at risk of developing HSCT-TMA. Subjects at risk of developing HSCT-TMA may benefit from administration of the agents referred to herein, in order to prevent HSCT-TMA or symptoms thereof. Risk factors for HSCT-TMA include the administration of medications used in the course of the HSCT conditioning, as well as several other patient characteristics as outlined below.

Whether the subject has undergone a myeloablative or non-myeloablative conditioning regimens can influence the risk of the subject developing HSCT-TMA. Subjects who have undergone myeloablative conditioning regimens are at a higher risk of developing HSCT-TMA relative to subjects which have not undergone myeloablative conditioning regimens. In some embodiments, the subject has undergone a myeloablative or non-myeloablative conditioning regimen, for example a myeloablative conditioning regimen.

Another risk factor is the prior treatment of the subject with calcineurin inhibitors (CNIs), which can increase the risk of developing HSCT-TMA [24]. CNIs are typically used to reduce inflammation and can be used to prevent and/or treat GVHD. These CNIs can either be used as part of a conditioning regimen or after the HSCT. Examples of CNIs are tacrolimus and pimecrolimus. In some embodiments, the subject has undergone treatment with one or more CNI, optionally wherein the CNI is tacrolimus.

Medications used in HSCT conditioning regimens can also increase the risk of the subject developing HSCT-TMA. Such medications include (i) alkylating antineoplastic agents, such as busulfan, (ii) cyclosporine, (iii) fludarabine; (iv) cisplatin; and/or (v) mammalian target of rapamycin (mTOR) inhibitors. In some embodiments, the subject has undergone treatment with one or more of: (i) alkylating antineoplastic agent(s), such as busulfan, (ii) cyclosporine, (iii) fludarabine; (iv) cisplatin; and/or (v) mammalian target of rapamycin (mTOR) inhibitor(s).

The presence of acute GVHD (aGVHD) in the subject can also increase the subject's risk of developing HSCT-TMA [24]. For example, the presence of grade II-IV aGVHD significantly increases the risk of the subject developing HSCT-TMA. In some embodiments, the subject has aGVHD.

In allogeneic HSCTs, the donor and recipient can contain matching or mismatching human leukocyte antigens (HLAs). These HLAs can include the HLA-A, HLA-B, HLA-C, HLA-DRB1, HLA-DQB1, and/or HLA-DPB1 loci. There is an increased risk of developing HSCT-TMA when the subject (i.e. the recipient) has received a HSCT with cells from a donor with mismatching HLAs [24]. In some embodiments, the subject has received a HSCT with cells from a donor with mismatching HLAs.

Patients that are at risk of developing HSCT-TMA also include those who are older, particularly those above the age of 35 [24]. In some embodiments, the subject is above the age of 35.

The presence of opportunistic infections in subjects that have undergone a HSCT increases the risk of developing HSCT-TMA [24]. Examples of such opportunistic infections include cytomegalovirus (CMV) infection. In some embodiments, the subject has one or more opportunistic infections, such as CMV infection.

Subjects having one or more of these risk factors are preferred, in terms of treatment or prevention of HSCT-TMA. In some embodiments a subject may have one or more of these risk factors but may not show clinical symptoms.

### Co-Occurrence with GVHD

In addition to HSCT-TMA, several other conditions are associated with HSCT. For example, GVHD is a systemic inflammatory syndrome that can also occur after allogeneic HSCT. The applicant has previously demonstrated that nomacopan is effective in treating and preventing aGVHD via complement inhibition [26]. LTB4 involvement in GVHD has recently been described [27], suggesting that LTB4 as an additional target for treating GVHD.

Accordingly, the therapeutics disclosed herein are particularly suited to treating the co-occurrence of GVHD and HSCT-TMA as the dual inhibition of both CS-cleavage and LTB4 activity can therefore effectively treat both (i) HSCT-TMA via complement inhibition; and (ii) GVHD via both complement inhibition and the inhibition of LTB4 activity.

The therapeutic agents disclosed herein may therefore be used in methods to treat both GVHD and HSCT-TMA in a subject. Alternatively stated, subjects having both HSCT-TMA and GVHD are preferred.

For example, the subject may be suffering from aGVHD, may have GVHD with one or more symptoms that are at stage +, ++, +++or +++++and/or the subject may have a clinical grading of I, II, III or IV, as summarised in reference [28]. These clinical stages and gradings are well known in the prior art, and are summarised in the tables below:

| | | Liver Findings | |
|---|---|---|---|
| | | (Bilirubin level, | |
| Stage | Skin Findings | mg/dL) | Gut Findings |
| + | Maculopapular rash | 2-3 | Diarrhea 500-1000 |
| | on <25% of body | | mL/d or persistent |
| | surface | | nausea |
| ++ | Maculopapular rash | 3-6 | Diarrhea 1000-1500 |
| | on 25-50% of body | | mL/d |
| | surface | | |
| ++ | Generalized | 6-15 | Diarrhea >1500 mL/d |
| | erythroderma | | |
| ++++ | Desquamation and | >15 | Pain with or without |
| | bullae | | ileus |

| | Stage | | | |
|---|---|---|---|---|
| | | | | Functional |
| Overall Grade | Skin | Liver | Gut | Impairment |
| 0 (None) | 0 | 0 | 0 | 0 |
| I Mild) | + to ++ | 0 | 0 | 0 |
| II (Moderate) | + to +++ | + | + | + |
| III (Severe) | ++ to +++ | ++ to +++ | ++ to +++ | ++ |
| IV (Life- | ++ to +++ | ++ to ++++ | ++ to ++++ | +++ |
| threatening) | | | | |

The outcome of the treatment the GVHD may be an improvement in the stage and/or grade of the GVHD. The subject suffering from GVHD may have tissue damage, e.g. internal (such as intestinal) tissue damage arising from the GVHD. As such, the outcome of the treatment may be a reduction in this tissue damage. Symptoms of GVHD can be measured by serum LDH. As such the outcome of the treatment may be a reduction in serum LDH e.g. as measured by standard methods known in the art.

The subject may have a reduced platelet count. As such the outcome of the treatment of GVHD may be an increase in platelet count, e.g. as measured by standard methods known in the art.

### Targeting the Complement System in the Treatment of HSCT-TMA

Complement activation has been identified in subjects with HSCT-TMA. Examples of complement abnormalities identified in HSCT-TMA subjects include anti-factor H antibodies as well as a high prevalence of a deletion that includes the genes encoding factor H-related proteins 1 and 3 [29]. Complement inhibitors, such as eculizumab, have previously been proposed to treat HSCT-TMA. There is some evidence that this treatment leads to resolution of the symptoms of TMA and improved survival [30, 31].

While treatment of HSCT-TMA with eculizumab has been proposed, several limitations are associated with its use. For example, as outlined below, subpopulations of patients are known to have C5-polymorphisms which reduce the binding of eculizumab to C5 and therefore render the patient resistant to treatment with eculizumab.

Furthermore, subjects diagnosed with, or are suspected of having HSCT-TMA may be treated with plasmapheresis, which functions to remove auto-antibodies. In addition to removing these auto-antibodies, any previously administered therapeutic antibodies, such as eculizumab, will also be removed. This thereby limits the utility of therapeutic antibodies in patients undergoing plasmapheresis. The inventors, however, have shown that the therapeutic agents disclosed herein do not suffer from such limitations, as they continue to work during and after plasmapheresis. In some embodiments therefore the subject is treated according to the invention before and/or after plasmapheresis (e.g. within 2, 3, 4, 5 days of plasmapheresis).

### Timing of Treatment

It can be advantageous to start treatment early after diagnosis or after disease onset. In a preferred embodiment of the invention the treatment of HSCT-TMA in subjects according to the invention starts not more than about 1 day from first diagnosis, not more than about 5 days from first diagnosis, not more than about 10 days from first diagnosis, not more than about 20 days from first diagnosis, not more than about 1 month from first diagnosis, not more than about 2 months from first diagnosis, not more than about 6 months from first diagnosis.

In a preferred embodiment of the invention the treatment of HSCT-TMA in subjects according to the invention is in subjects having not more than about 1 day disease duration, not more than about 5 days disease duration, not more than about 10 days disease duration, not more than about 20 days disease duration, not more than about 1 month disease duration, not more than about 2 months disease duration, not more than about 6 months disease duration.

### Outcomes of Administration

The subject may, as a result of the treatment, have reduced incidence of symptoms, alleviation of symptoms, inhibition or delay of occurrence or re-occurence of symptoms, or a combination thereof. Preferably the treatment gives rise to a reduction in the typical disease condition symptoms. For example, this may manifest as reducing one or more of the factors outlined above as diagnostic criteria.

The treatment may also result in a reduction in the amount or duration of a second HSCT-TMA treatment that is required.

The agent of the invention can be used in combination with other HSCT-TMA treatments. The combination of the agent of the invention with the other (referred to here as a “second”) HSCT-TMA treatment may be such that the amout of the second HSCT-TMA agent is reduced in comparison to the amount that is used in the absence of treatment with the agent of the invention, or the duration of the treatment with second HSCT-TMA agent is reduced in comparison to the duration of treatment that is used in the absence of treatment with the agent of the invenion. This is advantageous in view of the side effects of certain known treatments. Therefore, there is also provided a method of reducing the amount of a second HSCT-TMA treatment that is used for the treatment or reducing the duration of the treatment with a second HSCT-TMA treatment.

Preferably the second HSCT-TMA treatment is selected from: (i) a second complement inhibitor, such as an anti-CS antibody (e.g. eculizumab) or an anti-MASP2 antibody (e.g. OMS721); (ii) dose reduction or complete withdrawl of calcineurin inhibitors; (iii) plasma exchange (i.e. plasmapheresis); (iv) an anti-CD20 antibody, such as rituximab; (v) an anti-CD25 antibody, such as daclizumab; (vi) defibrotide; (vii) a vinca alkaloid, such as vincristine; (viii) a statin, such as pravastatin; (ix) transfusion of red blood cells and/or platelets; and (x) anti-hypertensives, such as thiazide diuretics, calcium channel blockers, ACE inhibitors, angiotensin II receptor antagonists (ARBs), and beta blockers. The second complement inhibitor can alternatively be selected from: LFG316 (Novartis, Basel, Switzerland, and MorphoSys, Planegg, Germany) or another antibody defined by the sequences of Table 1 in U.S. Pat. Nos. 8,241,628 and 8,883,158, ARC1905 (Ophthotech, Princeton, N.J. and New York, N.Y.), which is an anti-CS pegylated RNA aptamer, Mubodina® (Adienne Pharma & Biotech, Bergamo, Italy) (see, e.g., U.S. Pat. No. 7,999,081), ARC1005 (Novo Nordisk, Bagsvaerd, Denmark), SOMAmers (SomaLogic, Boulder, Colo.), SOB1002 (Swedish Orphan Biovitrum, Stockholm, Sweden), RA101348 (Ra Pharmaceuticals, Cambridge, Mass.), Aurin Tricarboxylic Acid (“ATA”), and anti siRNA (Alnylam Pharmaceuticals, Cambridge, Mass.).

When the agent of the invention and a second HSCT-TMA treatment are used, they may be administered or performed together or separately. The agent of the invention may be administered first and the second HSCT-TMA treatment may be administered or performed second, or vice versa.

Thus, where the agent of the invention is used in combination with one or more other HSCT-TMA treatments, e.g. in methods described as above, this can be described an agent which is a protein comprising amino acids 19 to 168 of the amino acid sequence in FIG. 2 (SEQ ID NO: 2) or a functional equivalent of this protein for use in a method of treating or preventing HSCT-TMA with a second HSCT-TMA treatment, or as a second HSCT-TMA treatment for use in a method of treating or preventing HSCT-TMA with an agent which is a protein comprising amino acids 19 to 168 of the amino acid sequence in FIG. 2 (SEQ ID NO: 2) or a functional equivalent of this protein.

Where the treatment gives rise to a reduction in the amount or duration of the second HSCT-TMA treatment, the reduction may be up to or at least 10, 20, 30, 40, 50, 60, 70, 80% compared to the amount of the second treatment that is used in the absence of the agent of the invention.

Various HSCT-TMA markers exist and are well known in the art. The outcome of the treatment may therefore be the shift in these markers towards, or to within, the parameters accepted as normal within the art. Such markers include hemolytic anaemia, red blood cell fragment count, thrombocytopenia, increased lactate dehydrogenase (LDH) levels, proteinuria and/or increased creatinine, hypertension, neurological symptoms and gastrointestinal (GI) bleeds. Proteinurea is defined as urine protein to creatinine ratio of greater than 2 mg/mg, and/or a random urinalysis protein concentration of ≥30 mg/dL.

Any reference to any reduction or increase is a reduction or increase in a disease parameter is compared to said subject in the absence of the treatment. Preferably, the parameter can be quantitated and where this is the case the increase or decrease is preferably statistically significant. For example, the increase or decrease may be at least 3, 5, 10, 15, 20, 30, 40, 50% or more compared to the parameter in the absence of treatment (e.g. before said treatment is started).

### Subjects

Preferred subjects, agents, doses and the like are as disclosed herein.

The subject to which the agent is administered in the practice of the invention is preferably a mammal, preferably a human. The subject to which the agent is administered is at risk of a HSCT-TMA or is a subject who has a HSCT-TMA. In some embodiments, the subject has elevated levels of terminal complement complex (sC5b9). In some embodiments, an elevated level of sC5b9 is above 244 ng/mL of serum. In some embodiments, the subject has evidence of complement deposition by histology.

Methods of the invention may also comprise one or more additional steps of (i) determining whether the subject is at risk of or has HSCT-TMA, (ii) determining the severity of the HSCT-TMA, which may be carried out before and/or after administration of nomacopan.

### Agent to be Used in the Invention

According to one embodiment of the invention, the agent is nomacopan itself or a functional equivalent thereof. In the following, the term “a nomacopan-type protein” is used as shorthand for “a protein comprising amino acids 19 to 168 of the amino acid sequence shown in FIG. 2 (SEQ ID NO: 2) or a functional equivalent thereof”.

Nomacopan was isolated from the salivary glands of the tick Ornithodoros moubata. Nomacopan is an outlying member of the lipocalin family and is the first lipocalin family member shown to inhibit complement activation. Nomacopan inhibits the classical, alternative and lectin complement pathways by binding to C5 and preventing its cleavage by C5 convertase into C5a and C5b, thus inhibiting both the production of C5a, which is an active (e.g. proinflammatory) peptide, and the formation of the MAC. Nomacopan has been demonstrated to bind to C5 and prevent its cleavage by C5 convertase in rat, mouse and human serum with an IC50 of approximately 0.02 mg/ml.

A nomacopan-type protein may thus comprise or consist of amino acids 19 to 168 of the amino acid sequence in FIG. 2 (SEQ ID NO: 2) or amino acids 1 to 168 of the amino acid sequence in FIG. 2 (SEQ ID NO: 2). The first 18 amino acids of the protein sequence given in FIG. 2 form a signal sequence which is not required for C5 binding or for LTB4 binding activity and so this may optionally be dispensed with, for example, for efficiency of recombinant protein production.

The nomacopan protein has been demonstrated to bind to C5 with a Kd of 1 nM, determined using surface plasmon resonance (SPR) [32]. Nomacopan-type peptides (e.g. functional equivalents of the nomacopan protein) preferably retain the ability to bind C5, conveniently with a Kd of less than 360 nM, more conveniently less than 300 nM, most conveniently less than 250 nM, preferably less than 200 nM, more preferably less than 150 nM, most preferably less than 100 nM, even more preferably less than 50, 40, 30, 20, or 10 nM, and advantageously less than 5 nM, wherein said Kd is determined using surface plasmon resonance, preferably in accordance with the method described in [32].

Nomacopan inhibits the classical complement pathway, the alternative complement pathway and the lectin complement pathway. Preferably, a nomacopan-type protein binds to C5 in such a way as to stabilize the global conformation of C5 but not directly block the C5 cleavage site targeted by the C5 convertases of the three activation pathways. Binding of nomacopan to C5 results in stabilization of the global conformation of C5 but does not block the convertase cleavage site. Functional equivalents of nomacopan also preferably share these properties.

C5 is cleaved by the C5 convertase enzyme (FIG. 1). The products of this cleavage include an anaphylatoxin C5a and a lytic complex C5b which promotes the formation of a complex of C5b, C6, C7, C8 and C9, also known as membrane attack complex (MAC). C5a is a highly pro-inflammatory peptide implicated in many pathological inflammatory processes including neutrophil and eosinophil chemotaxis, neutrophil activation, increased capillary permeability and inhibition of neutrophil apoptosis [33].

Monoclonal antibodies and small molecules that bind and inhibit C5 have been investigated for treating various diseases [34], in particular PNH, psoriasis, rheumatoid arthritis, systemic lupus erythematosus and transplant rejection. However, some of these monoclonal antibodies do not bind to certain C5 proteins from subjects with C5 polymorphisms, and are thus ineffective in these subjects [35]. Preferably, the nomacopan-type protein binds to and inhibits cleavage of not only wild-type C5 but also C5 from subjects with C5 polymorphisms (e.g. C5 polymorphisms that render treatment by eculizumab ineffective, or reduce the efficacy of treatment with eculizumab). The term “C5 polymorphism” includes any version of C5 which has been changed by insertion, deletion, amino acid substitution, a frame-shift, truncation, any of which may be single or multiple, or a combination of one or more of these changes compared to the wild-type C5. In a human subject, wild-type C5 is considered the C5 protein with accession number NP_001726.2; version GI: 38016947. Examples of C5 polymorphisms include polymorphisms at amino acid position 885, e.g. Arg885Cys (encoded by c.2653C>T) p.Arg885His (encoded by c.2654G>A) and Arg885Ser, which decrease the effectiveness of the monoclonal antibody eculizumab [35].

The ability of an agent to bind C5, including C5 from subjects with C5 polymorphisms, e.g. C5 polymorphisms that render treatment by eculizumab ineffective, or reduce the efficacy of treatment with eculizumab may be determined by standard in vitro assays known in the art, for example by surface plasmon resonance or western blotting following incubation of the protein on the gel with labelled C5. Preferably, the nomacopan-type protein binds C5, either wild-type and/or C5 from subjects with C5 polymorphisms, e.g. C5 polymorphisms that render treatment by eculizumab ineffective, or reduce the efficacy of treatment with eculizumab, with a Kd of less than 360 nM, more conveniently less than 300 nM, most conveniently less than 250 nM, preferably less than 200 nM, more preferably less than 150 nM, most preferably less than 100 nM, even more preferably less than 50, 40, 30, 20, or 10 nM, and advantageously less than 5 nM, wherein said Kd is determined using surface plasmon resonance, preferably in accordance with the method described in [32].

It may show higher, lower or the same affinity for wild-type C5 and C5 from subjects with C5 polymorphisms, e.g. C5 polymorphisms that render treatment by eculizumab ineffective, or reduce the efficacy of treatment with eculizumab.

The ability of a nomacopan-type protein to inhibit complement activation may also be determined by measuring the ability of the agent to inhibit complement activation in serum. For example, complement activity in the serum can be measured by any means known in the art or described herein.

The nomacopan-type protein may also be defined as having the function of inhibiting eicosanoid activity. Nomacopan has also been demonstrated to bind LTB4. Functional equivalents of the nomacopan protein may also retain the ability to bind LTB4 with a similar affinity as the nomacopan protein.

The ability of a nomacopan-type protein to bind LTB4 may be determined by standard in vitro assays known in the art, for example by means of a competitive ELISA between nomacopan and anti-LTB4 antibody competing for binding to labelled LTB4, by isothermal titration calorimetry or by fluorescence titration. Data obtained using fluorescence titration shows that nomacopan binds to LTB4 with a Kd of between 100 and 300 pM. For example, binding activity for LTB4 (Caymen Chemicals, Ann Arbor, Mich., USA) in phosphate buffered saline (PBS) can be quantified in a spectrofluorimeter e.g. a LS 50 B spectrofluorimeter (Perkin-Elmer, Norwalk, Conn., USA). This may be carried out by may be carried out as follows:

Purified 100 nM solutions of nomacopan, in 2 mL PBS were applied in a quartz cuvette (10 mm path length; Hellma, Mühlheim, Germany) equipped with a magnetic stirrer.

Temperature was adjusted to 20° C. and, after equilibrium was reached, protein Tyr/Trp fluorescence was excited at 280 nm (slit width: 15 nm). The fluorescence emission was measured at 340 nm (slit width: 16 nm) corresponding to the emission maximum. A ligand solution of 30 μM LTB4 in PBS was added step-wise, up to a maximal volume of 20 μL (1% of the whole sample volume), and after 30 s incubation steady state fluorescence was measured. For calculation of the KD value, data was normalized to an initial fluorescence intensity of 100%, the inner filter effect was corrected using a titration of 3 μM N-acetyl-tryptophanamide solution and data was plotted against the corresponding ligand concentration. Then, non-linear least squares regression based on the law of mass action for bimolecular complex formation was used to fit the data with Origin software version 8.5 (OriginLab, Northampton, Mass., USA) using a published formula (Breustedt et al., 2006) [36].

Nomacopan may bind LTB4 with an with a Kd of less than 1 nM, more conveniently less than 0.9 nM, most conveniently less than 0.8 nM, preferably less than 0.7 nM, more preferably less than 0.6 nM, most preferably less than 0.5 nM, even more preferably less than 0.4 nM, and advantageously less than 0.3 nM, wherein said Kd is determined using fluorescence titration, preferably in accordance with the method above. The nomacopan-type protein preferably shares these properties.

According to one embodiment of the invention, the nomacopan-type protein may bind to both C5 and to LTB4, e.g. to both wild-type C5 and C5 from subjects with C5 polymorphisms, e.g. C5 polymorphisms that render treatment by eculizumab ineffective, or reduce the efficacy of treatment with eculizumab, and to LTB4.

The nomacopan-type protein may thus act to prevent the cleavage of complement C5 by C5 convertase into complement C5a and complement C5b, and also to inhibit LTB4 activity. Using an agent which binds to both C5 and LTB4 is particularly advantageous when treating subjects which have co-occurrence of HSCT-TMA and GVHD, as outlined above.

Preferably, the agent of the invention is derived from a haematophagous arthropod. The term “haematophagous arthropod” includes all arthropods that take a blood meal from a suitable host, such as insects, ticks, lice, fleas and mites. Preferably, the agent is derived from a tick, preferably from the tick Ornithodoros moubata.

A functional equivalent of nomacopan may be a homologue or fragment of nomacopan which retains its ability to bind to C5, either wild-type C5 or C5 from a subject with a C5 polymorphism, and to prevent the cleavage of C5 by C5 convertase into C5a and C5b. The homologue or fragment may also retain its ability to bind LTB4.

Homologues include paralogues and orthologues of the nomacopan sequence that is explicitly identified in FIG. 2, including, for example, the nomacopan protein sequence from other tick species, including Rhipicephalus appendiculatus, R. sanguineus, R. bursa, A. americanum, A. cajennense, A. hebraeum, Boophilus microplus, B. annulatus, B. decoloratus, Dermacentor reticulatus, D. andersoni, D. marginatus, D. variabilis, Haemaphysalis inermis, Ha. leachii, Ha. punctata, Hyalomma anatolicum anatolicum, Hy. dromedarii, Hy. marginatum marginatum, Ixodes ricinus, I. persulcatus, I. scapularis, I. hexagonus, Argas persicus, A. reflexus, Ornithodoros erraticus, O. moubata moubata, O. m. porcinus, and O. savignyi.

The term “homologue” is also meant to include the equivalent nomacopan protein sequence from mosquito species, including those of the Culex, Anopheles and Aedes genera, particularly Culex quinquefasciatus, Aedes aegypti and Anopheles gambiae; flea species, such as Ctenocephalides felis (the cat flea); horseflies; sandflies; blackflies; tsetse flies; lice; mites; leeches; and flatworms. The native nomacopan protein is thought to exist in O. moubata in another three forms of around 18 kDa and the term “homologue” is meant to include these alternative forms of nomacopan.

Methods for the identification of homologues of the nomacopan sequence given in FIG. 2 will be clear to those of skill in the art. For example, homologues may be identified by homology searching of sequence databases, both public and private. Conveniently, publicly available databases may be used, although private or commercially-available databases will be equally useful, particularly if they contain data not represented in the public databases. Primary databases are the sites of primary nucleotide or amino acid sequence data deposit and may be publicly or commercially available. Examples of publicly-available primary databases include the GenBank database (http://www.ncbi.nlm.nih.gov/), the EMBL database (http://www.ebi.ac.uk/), the DDBJ database (http://www.ddbj.nig.acjp/), the SWISS-PROT protein database (http://expasy.hcuge.ch/), PIR (http://pir.georgetown.edu/), TrEMBL (http://www.ebi.ac.uk/), the TIGR databases (see http://www.tigr.org/tdb/index.html), the NRL-3D database (http://www.nbrfa.georgetown.edu), the Protein Data Base (http://www.rcsb.org/pdb), the NRDB database (ftp://ncbi.nlm.nih.gov/pub/nrdb/README), the OWL database (http://www.biochem.ucl.ac.uk/bsm/dbbrowser/OWL/) and the secondary databases PROSITE (http://expasy.hcuge.ch/sprot/prosite.html), PRINTS (http://iupab.leeds.ac.uk/bmb5dp/prints.html), Profiles (http://ulrec3.unil.ch/software/PFSCAN_form.html), Pfam (http://www.sanger.ac.uk/software/pfam), Identify (http://dna.stanford.edu/identify/) and Blocks (http://www.blocks.fhcrc.org) databases. Examples of commercially-available databases or private databases include PathoGenome (Genome Therapeutics Inc.) and PathoSeq (previously of Incyte Pharmaceuticals Inc.).

Typically, greater than 30% identity between two polypeptides (preferably, over a specified region such as the active site) is considered to be an indication of functional equivalence and thus an indication that two proteins are homologous. Preferably, proteins that are homologues have a degree of sequence identity with the nomacopan protein sequence identified in FIG. 2 (SEQ ID NO: 2) of greater than 60%. More preferred homologues have degrees of identity of greater than 70%, 80%, 90%, 95%, 98% or 99%, respectively with the nomacopan protein sequence given in FIG. 2 (SEQ ID NO:2). Percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1]. The % identity may be over the full length of the relevant reference sequence (e.g. amino acids 1-168 of SEQ ID NO:2 or amino acids 19-168 of SEQ ID NO:2).

Nomacopan-type proteins thus can be described by reference to a certain % amino acid sequence identity to a reference sequence e.g. amino acids 19-168 of FIG. 2, SEQ ID NO:2 or amino acids 1-168 of FIG. 2, SEQ ID NO:2 e.g. as a protein comprising or consisting of a sequence having at least 60%,70%, 80%, 90%, 95%, 98% or 99% identity to amino acids 19-168 of FIG. 2, SEQ ID NO:2 or amino acids 1-168 of FIG. 2, SEQ ID NO:2), Where the nomacopan-type protein comprises said sequence, the nomacopan-type protein may be a fusion protein (with e.g. a second protein, e,g. a heterologous protein). Suitable second proteins are discussed below.

In the various aspects and embodiments of this disclosure, the modified nomacopan polypeptides (e.g. nomacopan-type proteins) may differ from the unmodified nomacopan polypeptides in SEQ ID NO: 2 and SEQ ID NO: 4 by from 1 to 50, 2-45, 3-40, 4-35, 5-30, 6-25, 7-20, 8-25, 9-20, 10-15 amino acids, up to 1, 2, 3, 4, 5, 7, 8, 9, 10, 20, 30, 40, 50 amino acids. These may be substitutions, insertions or deletions but are preferably substitutions. Where deletions are made these are preferably deletion of up to 1, 2, 3, 4, 5, 7 or 10 amino acids, (e.g. deletions from the N or C terminus). Mutants thus include proteins containing amino acid substitutions, e.g. conservative amino acid substitutions that do not affect the function or activity of the protein in an adverse manner. This term is also intended to include natural biological variants (e.g. allelic variants or geographical variations within the species from which the nomacopan proteins are derived). Mutants with improved ability to bind wild-type C5 and/or C5 from subjects with a C5 polymorphism (e.g. C5 polymorphisms that render treatment by eculizumab ineffective, or reduce the efficacy of treatment with eculizumab) and/or LTB4 may also be designed through the systematic or directed mutation of specific residues in the protein sequence.

These modifications may be made to the nomacopan polypeptide as set out in SEQ ID NO: 2 and SEQ ID NO: 4 and the molecule will remain useful and will be considered to be a functional variant provided that the resulting modified nomacopan polypeptide retains LTB4 binding activity and C5 binding comparable with the nomacopan polypeptide as set out in SEQ ID NO: 2 and SEQ ID NO: 4, which can be determined e.g. using the tests referred to elsewhere herein (e.g. the binding to each of these is at least 80, 85, 90, 95% of the binding compared to the unmodified nomacopan polypeptide).

Given the requirement for functional variants to bind C5 and LTB4, when modification are made, certain residues should be excluded from modification. These include conserved cysteine resides. Other resides should be excluded from modification or, if substituted, should only be subject to conservative modification. These are the LTB4 binding residues and C5 binding residues as defined below. Given that the binding of LTB4 and C5 is relatively well understood it is possible to design a molecule that may have a percentage identity of around 65% to nomacopan but in which the changes are confined to residues which are not involved in C5 and LTB4 binding.

In some embodiments each of the six cysteine amino acids at positions 6, 38, 100, 128, 129, 150 of the mature nomacopan molecule (e.g. as set out in SEQ ID NO: 4 which corresponds to residues 19 to 168 of the full length protein including the signal sequence) is retained and at least five, ten or fifteen or each of the LTB4 binding residues and at least five, ten or fifteen or twenty or each of C5 binding residues set out below is retained or is subject to a conservative modification.

In some embodiments each of each of the six cysteine amino acids at positions 6, 38, 100, 128, 129, 150 of SEQ ID NO: 4 is retained and at least five, ten or fifteen or each of the LTB4 binding residues and at least five, ten or fifteen or twenty or each of C5 binding residues set out below is retained or is subject to a conservative modification, wherein up to 2, 3, 4, 5, 10, 15, 20 of the LTB4 and C5 binding residues are subject to a conservative modification. In some embodiments each of each of the six cysteine amino acids at positions 6, 38, 100, 128, 129, 150 of SEQ ID NO: 4 is retained and at least five, ten or fifteen or each of the LTB4 binding residues and at least five, ten or fifteen or twenty or each of C5 binding residues set out below is retained.

In some embodiments each of the six cysteine amino acids at positions 6, 38, 100, 128, 129, 150 of SEQ ID NO: 4 is retained and each of the LTB4 binding residues and each of C5 binding residues set out below is retained or is subject to a conservative modification.

In some embodiments each of each of the six cysteine amino acids at positions 6, 38, 100, 128, 129, 150 of SEQ ID NO: 4 is retained and each of the LTB4 binding residues and each of C5 binding residues set out below is retained or is subject to a conservative modification, wherein up to 2, 3, 4, 5, 10, 15, 20 of the C5 and/or LTB4 binding residues are subject to a conservative modification.

In some embodiments each of each of the six cysteine amino acids at positions 6, 38, 100, 128, 129, 150 of SEQ ID NO: 4 is retained and each of the LTB4 binding residues and each of C5 binding residues set out below is retained.

Modifications made outside of these regions may be conservative or non-conservative.

In each of these embodiments the spacing between these six cysteine amino acid residues is preferably retained to preserve the overall structure of the molecule (e.g. there molecule comprise six cysteine residues that are spaced relative to each other at a distance of 32 amino acids apart, 62 amino acids apart, 28 amino acids apart, 1 amino acid apart and 21 amino acids apart as arranged from the amino terminus to the carboxyl terminus of the sequence according to amino acids 1 to 168 of the amino acid sequence in FIG. 2).

### LTB4 Binding Residues

Resides that are thought to be involved in binding to LTB4 and are preferably retained in unmodified form or are subject to conservative changes only in the sequence of any molecule that is modified relative to SEQ ID NO:2 or SEQ ID NO:4 are Phe18, Tyr25, Arg36, Leu39, Gly41, Pro43, Leu52, Va154, Met56, Phe58, Thr67, Trp69, Phe71, Gln87, Arg89, His99, His101, Asp103, and Trp115 (numbering according to SEQ ID NO:4).

### C5 Binding Residues

Resides that are thought to be involved in binding to C5 are preferably retained in unmodified form in the sequence of any molecule that is modified relative to SEQ ID NO:2 or SEQ ID NO:4 are Va126, Va128, Arg29, Ala44, Gly45, Gly61, Thr62, Ser97, His99, His101, Met 114, Met 116, Leu117, Asp118, Ala119, Glyl20, Glyl21, Leu122, Glu123, Va1124, Glu125, Glu127, His146, Leu147 and Asp 149 (numbering according to SEQ ID NO:4).

### LTB4 and/or C5 Binding Residues

There are two histidine residues involved in both LTB4 and C5 binding, His99 and His101. The list of residues involved in LTB4 and/or C5 binding is therefore Phe18, Tyr25, Va126, Va128, Arg29, Arg36, Leu39, Gly41, Pro43, Ala44, Gly45, Leu52, Va154, Met56, Phe58, Gly61, Thr62, Thr67, Trp69, Phe71, Gln87, Arg89, Ser97, His99, His101, Asp103, Met 114, Trp115, Met 116, Leu117, Asp118, Ala119, Glyl20, Glyl21, Leu122, Glu123, Va1124, Glu125, Glu127, His146, Leu147 and Asp 149 (numbering according to SEQ ID NO:4).

Functional equivalents of nomacopan include fragments of the nomacopan protein providing that such fragments retain the ability to bind wild-type C5 and/or C5 from subjects with a C5 polymorphism (e.g. C5 polymorphisms that render treatment by eculizumab ineffective, or reduce the efficacy of treatment with eculizumab) and/or LTB4. Fragments may include, for example, polypeptides derived from the nomacopan protein sequence (or homologue) which are less than 150 amino acids, less than 145 amino acids, provided that these fragments retain the ability to bind to complement wild-type C5 and/or C5 from subjects with a C5 polymorphism (e.g. C5 polymorphisms that render treatment by eculizumab ineffective, or reduce the efficacy of treatment with eculizumab) and/or LTB4. Fragments may include, for example, polypeptides derived from the nomacopan protein sequence (or homologue) which are at least 150 amino acids, at least 145, amino acids, provided that these fragments retain the ability to bind to complement wild-type C5 and/or C5 from subjects with a C5 polymorphism (e.g. C5 polymorphisms that render treatment by eculizumab ineffective or reduce the efficacy of treatment with eculizumab) and/or LTB4.

Any functional equivalent or fragment thereof preferably retains the pattern of cysteine residues that is found in nomacopan. For example, said functional equivalent comprises six cysteine residues that are spaced relative to each other at a distance of 32 amino acids apart, 62 amino acids apart, 28 amino acids apart, 1 amino acid apart and 21 amino acids apart as arranged from the amino terminus to the carboxyl terminus of the sequence according to amino acids 1 to 168 of the amino acid sequence in FIG. 2 (SEQ ID NO:2). Exemplary fragments of nomacopan protein are disclosed in SEQ ID NO: 4, SEQ ID NO: 6, SEQ ID NO: 8, SEQ ID NO: 10, SEQ ID NO: 12, SEQ ID NO: 14. The DNA encoding the corresponding fragments are disclosed in SEQ ID NO: 3, SEQ ID NO: 5, SEQ ID NO: 7, SEQ ID NO: 9, SEQ ID NO: 11, SEQ ID NO: 13.

Included as such fragments are not only fragments of the O. moubata nomacopan protein that is explicitly identified herein in FIG. 2, but also fragments of homologues of this protein, as described above. Such fragments of homologues will typically possess greater than 60% identity with fragments of the nomacopan protein sequence in FIG. 2, although more preferred fragments of homologues will display degrees of identity of greater than 70%, 80%, 90%, 95%, 98% or 99%, respectively with fragments of the nomacopan protein sequence in FIG. 2. Preferably such fragment will retain the cysteine spacing referred to above. Fragments with improved properties may, of course, be rationally designed by the systematic mutation or fragmentation of the wild type sequence followed by appropriate activity assays. Fragments may exhibit similar or greater affinity for C5, either the wild-type or polymorphic variant of C5 or both, and/or LTB4 as nomacopan. These fragments may be of a size described above for fragments of the nomacopan protein.

As discussed above, nomacopan-type proteins preferably bind to both wild-type C5 and/or C5 from subjects with a C5 polymorphism (e.g. C5 polymorphisms that render treatment by eculizumab ineffective, or reduce the efficacy of treatment with eculizumab) and LTB4. Any substitutions are preferably conservative substitutions, for example according to the following Table. Amino acids in the same block in the second column and preferably in the same line in the third column may be substituted for each other:

| | | |
|---|---|---|
| Aliphatic | Non-polar | G A P |
| | | I L V |
| | Polar - uncharged | C S T M |
| | | N Q |
| | Polar - charged | D E |
| | | K R |
| Aromatic | | H F W Y |

A functional equivalent used according to the invention may be a fusion protein, obtained, for example, by cloning a polynucleotide encoding the nomacopan protein or a functionally equivalent in frame to the coding sequences for a heterologous protein sequence. The term “heterologous”, when used herein, is intended to designate any polypeptide other than the nomacopan protein or its functional equivalent. Example of heterologous sequences that can be comprised in the soluble fusion proteins either at N- or at C-terminus, are the following: extracellular domains of membrane-bound protein, immunoglobulin constant regions (Fc region), PAS or XTEN or similar unstructured polypeptides, multimerization domains, domains of extracellular proteins, signal sequences, export sequences, or sequences allowing purification by affinity chromatography. Many of these heterologous sequences are commercially available in expression plasmids since these sequences are commonly included in the fusion proteins in order to provide additional properties without significantly impairing the specific biological activity of the protein fused to them [37]. Examples of such additional properties are a longer lasting half-life in body fluids (e.g. resulting from the addition of an Fc region or PASylation [38]), the extracellular localization, or an easier purification procedure as allowed by a tag such as a histidine, GST, FLAG, avidin or HA tag. Fusion proteins may additionally contain linker sequences (e.g. 1-50 amino acids in length, such that the components are separated by this linker.

Fusion proteins are thus examples of proteins comprising a nomacopan-like protein, and include by way of specific example a protein comprising a PAS sequence and a nomacopan-type protein sequence. PAS sequences are described e.g. in [38], and EP2173890, with a PASylated nomacopan molecule being described in Kuhn et al [39]. PASylation describes the genetic fusion of a protein with conformationally disordered polypeptide sequences composed of the amino acids Pro, Ala, and/or Ser. This is a technology developed by XL Protein (http://xl-protein.com/) and provides a simple way to attach a solvated random chain with large hydrodynamic volume to the protein to which it is fused. The polypeptide sequence adopts a random coil structure. The apparent molecular weight of the resulting fusion protein is thus much larger than the actual molecular weight of the fusion protein. This greatly reduces clearance rates by kidney filtration in biological systems. Appropriate PAS sequences are described in EP2173890, as well as [38]. Any suitable PAS sequence may be used in the fusion protein. Examples include an amino acid sequence consisting of at least about 100 amino acid residues forming a random coil conformation and consisting of or consisting essentially of alanine, serine and proline residues (or consisting of or consisting essentially of proline and alanine residues). This may comprise a plurality of amino acid repeats, wherein said repeats consist of or consist essentially of Ala, Ser, and Pro residues (or proline and alanine residues) and wherein no more than 6 consecutive amino acid residues are identical. Proline residues may constitute more than 4% and less than 40% of the amino acids of the sequence. The sequence may comprise an amino acid sequence selected from:

(SEQ ID NO: 15)
ASPAAPAPASPAAPAPSAPA;
(SEQ ID NO: 16)
AAPASPAPAAPSAPAPAAPS;
(SEQ ID NO: 17)
APSSPSPSAPSSPSPASPSS,
(SEQ ID NO: 18)
SAPSSPSPSAPSSPSPASPS,
(SEQ ID NO: 19)
SSPSAPSPSSPASPSPSSPA,
(SEQ ID NO: 20)
AASPAAPSAPPAAASPAAPSAPPA
and
(SEQ ID NO: 21)
ASAAAPAAASAAASAPSAAA

or circular permuted versions or multimers of these sequences as a whole or parts of these sequences. There may, for example be 5-40, 10-30, 15-25, 18-20 preferably 20-30 or 30 copies of one of the repeats present in the PAS sequence, i.e. one of SEQ ID NOs 15-21, preferably 15. Preferably the PAS sequence comprises or consists of 30 copies of SEQ ID NO:15. Preferably the PAS sequence is fused to the N terminus of the nomacopan-type protein (directly or via a linker sequence) and in certain preferred embodiments the nomacopan-type protein may comprise or consist of amino acids 19-168 of SEQ ID NO:2 (e.g. the fusion protein comprises (a) a PAS sequence consisting of 30 copies of SEQ ID NO:15 and (b) amino acids 19-168 of SEQ ID NO:2, wherein (a) is fused to the N terminus of (b) directly or via a linker sequence). An exemplary sequence is provided in FIG. 6 and SEQ ID NO:22.

Fusion proteins may additionally contain linker sequences (e.g. 1-50, 2-30, 3-20, 5-10, 2-4, 3-5, 1, 2, 3, 4, 5, 6, 7, 8, 9, 10 or up to 1, 2, 3, 4, 5, 6, 7, 8, 9, 10 amino acids in length), such that the components are separated by this linker. In one embodiment the linker sequence can be a single alanine residue.

In the present “PAS-nomacopan” is intended to refer to a functional equivalent of nomacopan that is PASylated, e.g. as described above. The precise sequence of the tested PAS-nomacopan molecule in Examples 1 and 2 is set out in FIG. 6 and SEQ ID NO:22. PAS-nomacopan has the advantage that its longer half-life allows less frequent administration, which is more convenient for patients. PAS-nomacopan thus combines the advantages of nomacopan, in that it inhibits both the C5 and the LTB4 dependent pathways, yet can be administered less frequently than nomacopan thus providing an administration advantage.

The protein and functional equivalents thereof, may be prepared in recombinant form by expression in a host cell. Such expression methods are well known to those of skill in the art and are described in detail by [40] and [41]. Recombinant forms of the nomacopan protein and functional equivalents thereof are preferably unglycosylated. Preferably the host cell is E.coli.

The nomacopan protein and functional equivalents thereof, are preferably in isolated form, e.g. separated from at least one component of the host cell and/or cell growth media in which it was expressed. In some embodiments, the nomacopan protein or functional equivalent thereof is purified to at least 90%, 95%, or 99% purity as determined, for example, by electrophoresis or chromatography. The proteins and fragments of the present invention can also be prepared using conventional techniques of protein chemistry. For example, protein fragments may be prepared by chemical synthesis. Methods for the generation of fusion proteins are standard in the art and will be known to the skilled reader. For example, most general molecular biology, microbiology recombinant DNA technology and immunological techniques can be found in [40] or [42].

According to a further embodiment of the invention, the agent may be a nucleic acid molecule encoding the nomacopan-type protein. For example, gene therapy may be employed to effect the endogenous production of the nomacopan-type protein by the relevant cells in the subject, either in vivo or ex vivo. Another approach is the administration of “naked DNA” in which the therapeutic gene is directly injected into the bloodstream or into muscle tissue.

Preferably, such a nucleic acid molecule comprises or consists of bases 55 to 507 of the nucleotide sequence in FIG. 2 (SEQ ID NO: 1). This nucleotide sequence encodes the nomacopan protein in FIG. 2 without the signal sequence. The first 54 bases of the nucleotide sequence in FIG. 2 encode the signal sequence which is not required for complement inhibitory activity or LTB4 binding activity. Alternatively, the nucleic acid molecule may comprise or consist of bases 1 to 507 of the nucleic acid sequence in FIG. 2, which encodes the protein with the signal sequence.

### Modes of Administration

Nomacopan-type proteins do not require a medical professional for administration to be carried out, and these molecules are rapidly absorbed. In contrast, many recombinant antibodies are absorbed very slowly or cannot be administered by subcutaneous injection or other routes of administration and as a result need to be infused over long periods (e.g. intravenously). The administration of such molecules therefore requires a medical professional. Thus, nomacopan-type proteins also possess the advantage of being easier to administer than other agents that require infusion.

The agent is administered in a therapeutically or prophylactically effective amount. The term “therapeutically effective amount” refers to the amount of agent needed to treat the HSCT-TMA. In this context, “treating” includes reducing the severity of the disorder.

The term “prophylactically effective amount” used herein refers to the amount of agent needed to prevent the relevant condition, e.g. HSCT-TMA. In this context, “preventing” includes reducing the severity of the disorder, e.g. if the presence of the disorder is not detected before the administration of the agent is commenced.

The reduction or improvement is relative to the outcome without administration or the agent as described herein. The outcomes are assessed according to the standard criteria used to assess such patients, such as the diagnostic criteria described above. To the extent that this can be quantitated, there is a reduction or improvement of at least 10, 20, 30, 40, 50, 60, 70, 80, 90, 100% in the relative criteria.

Preferably, the dose, calculated on the basis of the nomacopan molecule is from 0.1 mg/kg/day to 10 mg/kg/day (mass of drug compared to mass of patient), e.g. 0.2-5, 0.25-2, or 0.1-1 mg/kg/day. In some embodiments, the dose of nomacopan is from 0.25 mg/kg/day to 2 mg/kg/day. As fusion proteins (e.g. as discussed herein) are larger than the nomacopan molecule an equivalent molar amount could be used for such proteins. Thus, for a functional equivalent of nomacopan, an equivalent molar amount of the dose referred to above can be used. For example for a fusion protein comprising nomacopan and a PAS portion of about 600 amino acids, or a PAS portion as defined herein, e.g. PAS-nomacopan) an equivalent molar amount of 0.1 mg/kg/day is 0.4 mg/kg/day, so the dose could be 0.4 mg/kg/day to 40 mg/kg/day (mass of drug compared to mass of patient), e.g. 0.8-20, 1-8, or 0.4-4 mg/kg/day. Alternatively, and to account for the longer half-life of these fusion proteins, greater amounts can be given per dose, and the dose administered less often, e.g. 40 mg-2 g, 50 mg-1.5 g, 75 mg-1 g, over the course of one week, e.g. with administration being e.g. one or twice per week.

The therapeutically or prophylactically effective amount can additionally be defined in terms of the inhibition of terminal complement, for example, an amount that means that terminal complement activity (TCA) is reduced by at least 10, 20, 30, 40, 50, 60, 70, 80, 90, 91, 92, 93, 94, 95, 96, 97, 98, 99, 100%, compared to terminal complement activity in the absence of treatment. Dose and frequency may be adjusted in order to maintain terminal complement activity at the desired level, which may be, for example 10% or less, for example 9, 8, 7, 6, 5, 4, 3, 2, 1% or less compared to terminal complement activity in the absence of treatment.

The therapeutically or prophylactically effective amount can additionally be defined in terms of the reduction of LTB4 levels in plasma, for example, an amount that means that the LTB4 level in plasma is reduced by at least 10, 20, 30, 40, 50, 60, 70, 80, 90, 91, 92, 93, 94, 95, 96, 97, 98, 99, 100%, compared to the LTB4 level in plasma in the absence of treatment or which causes LTB4 levels to be within a certain range of the normal levels (e.g. 90-110% of normal, 85-115% of normal). Dose and frequency may be adjusted in order to maintain the LTB4 level in plasma at the desired level, which may be, for example 90%, 80%, 70%, 60%, 50%, 40%, 30%, 20%, 10% or less, for example 9, 8, 7, 6, 5, 4, 3, 2, 1% or less compared to the LTB4 level in plasma in the absence of treatment or which is within a certain range of the normal levels (e.g. 90-110% of normal, 85-115% of normal). LTB4 levels may be determined by routine methods (e.g. immunoassays, see e.g. the commercially available R&D Systems assay based on a sequential competitive binding technique [43]).

Where a dose is given, this relates to a dose of the agent which is a protein or functional equivalent thereof. Appropriate doses for an agent which is a nucleic acid molecule may be used to give rise to these levels. Doses may vary to account for the presence of non-active protein present (e.g. PAS-nomacopan with a 600 amino acid PAS portion has a higher molecular weight than nomacopan so an equivalent molar amount would take this into account). An equivalent molar amount of any dose provided for nomacopan may be used for any nomacopan functional equivalent thereof which contains additional sequence. The equivalent molar amount can be calculated using routine methods.

Terminal complement activity can be measured by standard assays known in the art, e.g. using the Quidel CH₅₀ haemolysis assay and the sheep red blood cell lytic CH50 assay.

The frequency with which the dose needs to be administered will depend on the half-life of the agent involved. The nomacopan protein or a functional equivalent thereof, may be administered e.g. on a twice daily basis, daily basis, or every two, three, four days, five, six, or seven, days or more e.g. twice daily or on a daily basis). Extended half-life versions, e.g. PASylated nomacopan molecules could be administered less frequently (e.g. every two, three, four days, five, six, seven, 10, 15 or 20 days or more, e.g. once daily or every two or more days, or every week)

The exact dosage and the frequency of doses may also be dependent on the patient's status at the time of administration. Factors that may be taken into consideration when determining dosage include the need for treatment or prophylaxis, the severity of the disease state in the patient, the general health of the patient, the age, weight, gender, diet, time and frequency of administration, drug combinations, reaction sensitivities and the patient's tolerance or response to therapy. The precise amount can be determined by routine experimentation, but may ultimately lie with the judgement of the clinician.

The dosage regimen may also take the form of an initial “ablating regimen” followed by one or more subsequent doses (e.g. maintenance dose). In general, the ablating regimen will be greater than the subsequent dose(s). By way of example for nomacopan this may be an ablating regimen of 0.6-1.2 mg/kg, then 0.3-0.6 mg/kg 8-18, 10-14, or 11-13 hours (e.g. about 12 hours) later, followed by a maintenance dose of 0.45-0.9 mg/kg, which may be administered e.g. once daily.

For PASylated versions (e.g. PAS-nomacopan, e.g. as described elsewhere herein a suitable regimen may be an ablating regimen of 6-12 mg/kg (e.g. 600 mg), then 6-12 mg/kg (e.g. 600 mg) 3-10, 4-8, 5-7, e.g. about 7 days later, followed by a maintenance dose of 4-8 mg/kg (e.g. 400 mg), which may be administered e.g. once daily.

The ablating dose or doses may be at least 1.5, 2, or 5 times greater than the maintenance dose. The ablating dose may be administered as a single dose, or as one or more doses in a particular time frame (e.g. two doses). Typically, the loading dose will be 1, 2, 3, 4 or 5 doses administered in a single 24 hour period (or a single week for an extended half-life version). The maintenance dose may be a lower dose that is repeated at regular intervals. The maintenance dose may be repeated at intervals, such as every 12, 24, or 48 hours (or every week, or every two weeks for an extended half-life version). The precise regimen can be determined by routine experimentation, but may ultimately lie with the judgement of the clinician. The maintenance dose may be at least 20, 30, 40, 50, 60, 70, 80, 90 or 100% of the initial ablating dose, or up to 20, 30, 40, 50, 60, 70, 80, 90 or 100% of the initial ablating dose.

In a further embodiment the same dose is used throughout the course of treatment (e.g. daily or twice daily or weekly).

The agent will generally be administered in conjunction with or in a pharmaceutically acceptable carrier. The term “pharmaceutically acceptable carrier”, in general will be a liquid or but may include other agents provided that the carrier does not itself induce toxicity effects or cause the production of antibodies that are harmful to the individual receiving the pharmaceutical composition. Pharmaceutically acceptable carriers may e.g. contain liquids such as water, saline, glycerol, ethanol or auxiliary substances such as wetting or emulsifying agents, pH buffering substances and the like. The pharmaceutical carrier employed will thus vary depending on the route of administration. A thorough discussion of pharmaceutically acceptable carriers is available in [44]. In a preferred embodiment the agent is administered in a liquid, e.g. in a solution in water or PBS.

The agent may optionally be delivered using colloidal delivery systems (e.g. liposomes, nanoparticles or microparticles (e.g. as discussed in [45])). Advantages of these carrier systems include protection of sensitive proteins, prolonged release, reduction of administration frequency, patient compliance and controlled plasma levels.

Liposomes (e.g. comprising phospholipids of synthetic and/or natural origin) may e.g. be 20 nm 100 or 200 micrometers, e.g. small unilamellar vesicles (25-50 nm), large unilamellar vesicles (100-200 nm), giant unilamellar vesicles (1-2 μn) or multilamellar vesicles (MLV; 1 μm-2 μm).

Nanoparticles (colloidal carriers with size ranging from 10 to 1000 nm) can be fabricated from lipids, polymers or metal. Polymeric nanoparticles may be made from natural or synthetic polymers (e.g. chitosan, alginate, PCL, polylactic acid (PLA), poly (glycolide), PLGA and may be generated as nanospheres (molecules are uniformly distributed into polymeric matrix) or nanocapsules (carrying drug molecules confined within a polymeric membrane).

Microparticles e.g. made of starch, alginate, collagen, poly (lactide-co-glycolide) (PLGA), polycaprolactones (PCL) can also be used.

Hydrogels may alternatively or additionally be present.

For larger molecular weight molecules, e.g. fusion proteins additional excipients such as hyaluronidase may also be used, e.g. to allow administration of larger volumes (e.g. 2-20ml).

The agent is preferably delivered by subcutaneous injection or injection into the synovial joint fluid. Subcutaneous injection is preferred in view of the ease of administration for the subject. In some embodiments this is via once or twice daily subcutaneous injection.

Preferably the course of treatment is continued for at least 1, 2, 3, 4, 5 or 6 weeks, or at least 1, 2, 3, 4, 5 or 6 months or at least 1, 2, 3, 4, 5 or 6 years. The course of treatment is preferably continued at least until the subject's symptoms have reduced. The course of treatment may thus be administration of the agent (e.g. daily, every other day or weekly) for at least 1, 2, 3, 4, 5, 6, 7, 8, 9, 10, 15, 20, 25, 30, 35, 40 weeks.

The maintenance dose (e.g. a single daily or weekly maintenance dose) may remain constant throughout the course of treatment) or the maintenance dose (e.g. a daily maintenance dose) may be modified (e.g. increased or decreased) during the course of treatment. The maintenance dose may be modified in order to maintain terminal complement activity and plasma LTB4 levels at a desired level, e.g. terminal complement activity at 10% or less compared to serum from said patient in the absence of treatment or compared to normal control serum and/or plasma LTB4 levels at 90% or less compared to plasma from said patient in the absence of treatment, or to attain plasma LTB4 levels that are within a certain range of the normal levels (e.g. 90-110% of normal, 85-115% of normal). The or each maintenance dose may be continued for at least 1, 2, 3, 4, 5, 6, 7, 8, 9, 10 weeks, e.g. daily for at least 1, 2, 3, 4, 5, 6, 7, 8, 9, 10 weeks. The maintenance dose may be decreased as the subject's symptoms improve. The amount of agent or the frequency with which the agent is administered may be decreased as the subject's symptoms improve.

There may thus be an initial ablating dose or regimen, followed by an initial maintenance dose (e.g. a daily or weekly initial maintenance dose) which may be a maintenance dose as defined above, and one or more further maintenance doses (e.g. a daily or weekly further maintenance dose), e.g. at least 2, 3, 4, 5 further maintenance doses.

The invention thus further comprises a method of treating or preventing a HSCT-TMA in a subject, comprising administering to the subject an initial ablating dose or regimen of the agent as defined above, and then administering maintenance doses (e.g. daily or weekly maintenance doses) of the agent as defined above, wherein there is an initial maintenance dose and one or more further maintenance doses.

The invention thus further comprises an agent as defined above for use in a method of treating or preventing a HSCT-TMA in a subject, the method comprising administering to the subject an initial ablating dose or regimen of the agent as defined above, and then administering maintenance doses (e.g. daily or weekly maintenance doses) of the agent as defined above, wherein there is an initial maintenance dose and one or more further maintenance doses.

The one or more further maintenance doses may be determined by testing the terminal complement activity in the subject (e.g. in a biological sample from the subject) or plasma LTB4 level, and determining the further maintenance dose on the basis of the level of terminal complement activity and/or plasma LTB4 level and/or testing the subject's symptoms and determining the further maintenance dose on the basis of the symptoms. The method may optionally further comprise administering said further maintenance dose. Said further dose may be calculated to be at a level that maintains terminal complement activity at the desired level.

Where a biological sample is taken, this may be blood, e.g. a whole blood, plasma or a serum sample. The method optionally further comprises the step of taking the sample, and further optionally comprises the step of determining the TCA of the sample and/or the step of determining the plasma LTB4 level

The one or more further maintenance doses may be determined by testing the terminal complement activity in the subject (e.g. in a biological sample) and/or plasma LTB4 level, and determining the further maintenance dose on the basis of the level of terminal complement activity and/or plasma LTB4 level, and/or testing the subject's symptoms and determining the further maintenance dose on the basis of the symptoms. The method may optionally further comprise administering said further maintenance dose. Said further dose may be calculated to be at a level that maintains terminal complement activity and/or plasma LTB4 level at the desired level.

In certain aspects, the desired complement activity level is 10% or less compared to serum from said subject in the absence of treatment or compared to normal control serum and/or plasma LTB4 level is 90% or less compared to plasma from said patient in the absence of treatment, and/or plasma LTB4 levels are within a certain range of the normal levels (e.g. 90-110% of normal, 85-115% of normal).

In certain aspects, if the TCA and/or plasma LTB4 is higher than the desired level the maintenance dose is increased, and optionally wherein if TCA is less than 5, 4, 3, 2, 1% and/or LTB4 plasma levels are 90% or less compared to plasma from said patient in the absence of treatment (or plasma LTB4 levels are within a certain range of the normal levels (e.g. 90-110% of normal, 85-115% of normal) the dose is maintained or decreased.

In certain aspects, if the symptoms deteriorate the maintenance dose is increased, and optionally wherein if the symptoms improve the dose is maintained or decreased.

In some embodiments the subject is tested within one month of initiating the treatment, within two weeks of initiating the treatment, within a week of initiating the treatment. In other embodiments the subject is tested once a day or at least once a day, once a week, or at least once a week, once every two weeks or at least once every two weeks, once a month or once every two months.

The dosage regimen may also take the form of fixed dose not dependent on the weight of the subject being treated. The fixed dose may be administered as a single dose, or as one or more doses in a particular time frame. The fixed dose can be lmg-500 mg of nomacopan (e.g. SEQ ID NO: 4) for typical human patients (e.g. those between 50 kg and 100 kg in weight). The molecular weight of nomacopan-type proteins can be used to calculate equivalent fixed doses of functionally equivalent agents. In some embodiments, the fixed dose is between 1 mg-400 mg, 1 mg-300 mg, 1 mg-200 mg, 1 mg-100 mg, 1 mg-50 mg, 1 mg-20 mg, 1 mg-10 mg, 5 mg-80 mg, 5 mg-50 mg, 10 mg-60 mg, 10 mg-50 mg, 20 mg-50 mg, 20 mg-40 mg or 25 mg-35 mg of nomacopan (e.g. SEQ ID NO: 4) or the molar equivalent of a nomacopan-type protein. Preferably the fixed dose is 30 mg, or 45 mg of nomacopan (SEQ ID NO: 4) or the molar equivalent of a nomacopan-type protein. Typically, the fixed dose will be 1, 2, 3, 4 or 5 doses administered in a single 24 hour period. The fixed dose may be repeated at intervals, such as every 3, 4, 6, 8, 12, 24, or 48 hours. The precise regimen can be determined by routine experimentation, but may ultimately lie with the judgement of the clinician.

### BRIEF DESCRIPTION OF FIGURES

FIG. 1: Schematic diagram of classical and alternative pathways of complement activation. Anaphylatoxins enclosed in starbursts.

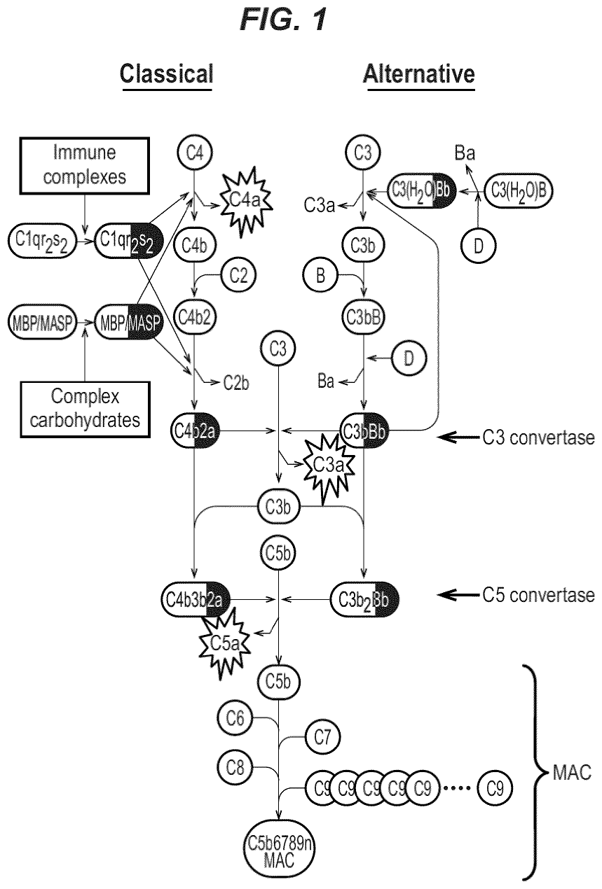

FIG. 2A: Primary sequence of nomacopan. Signal sequence underlined. Cysteine residues in bold type. Nucleotide and amino acid number indicated at right.

FIG. 2B: Examples of nomacopan variants.

FIG. 3: Table showing the clinical progression of two HSCT-TMA patients that were treated with nomacopan.

FIG. 4: Complement activity in HSCT-TMA patients after treatment with nomacopan.

FIG. 5: Level of free nomacopan in the serum of HSCT-TMA patients after treatment with nomacopan.

FIG. 6: PAS-nomacopan sequence.

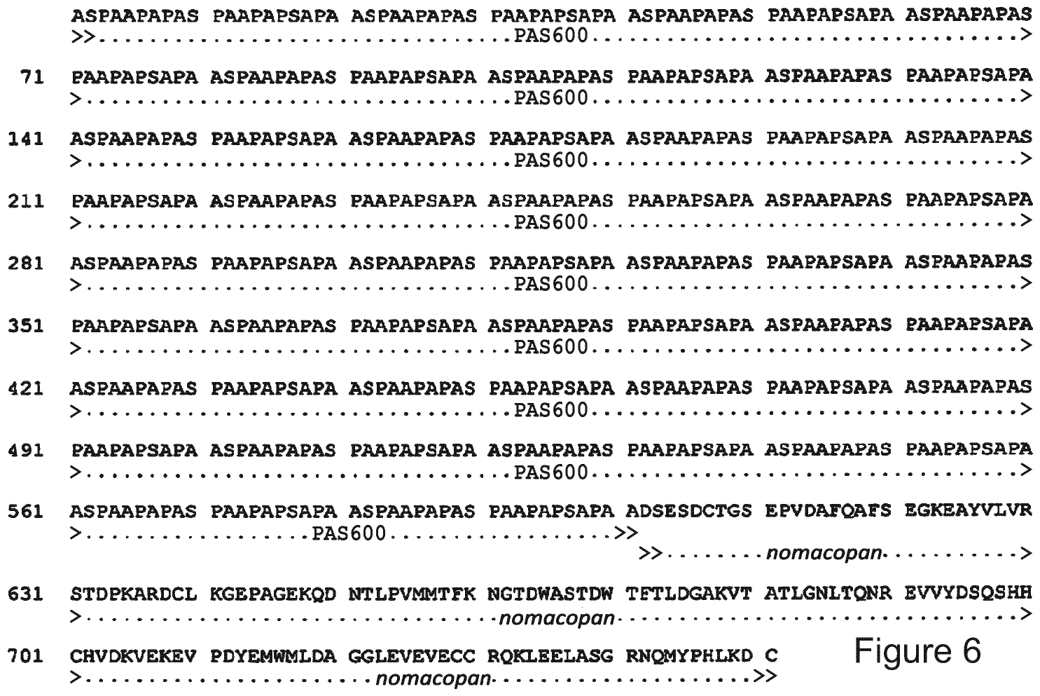

### EXAMPLE

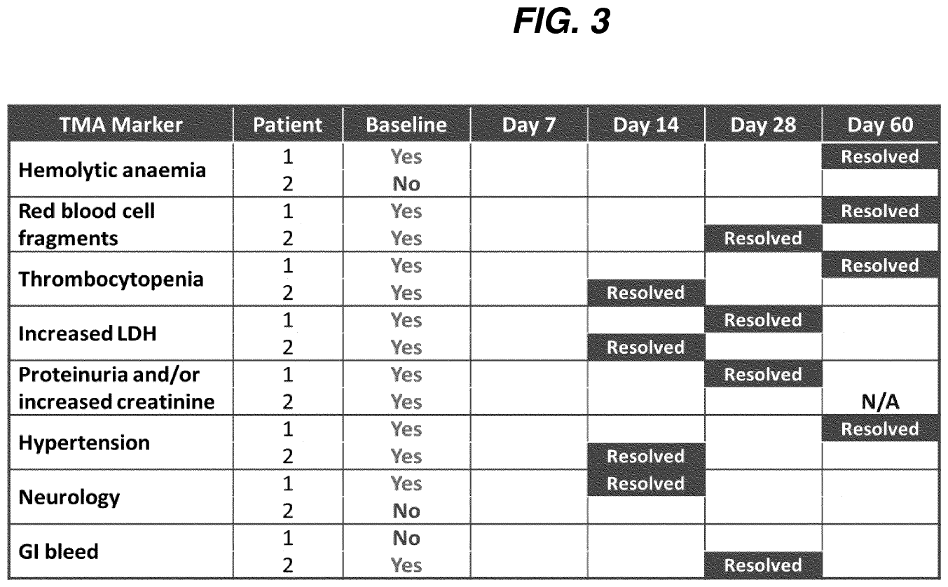

Two patients with HSCT-TMA were treated with nomacopan, and the results are shown in FIG. 3. This was part of a named patient program in the UK.

The first patient (patient 1 in FIG. 3) was diagnosed with HSCT-TMA in early June 2018 having had a HSCT transplant for treatment of acute lymphoblastic leukemia on 19 Jan. 2018. At presentation, the patient had no signs of active infection or GVHD but had tremor and abdominal pain, skin lesions, hypertension, edema, weight gain, and blood abnormalities including thrombocytopenia, anemia, elevated LDH, red blood cell fragments. She also had proteinuria and erythrocyturia, very high soluble terminal complement complex (sC5b9) levels (692 ng/mL) and neurological impairment. The patient received nomacopan dosing described in Table 1 starting on the 8 Jun. 2018 and resolved her symptoms of HSCT-TMA within 63 days of initiating nomacopan (FIG. 3), She developed no infections or GVHD and was alive and well more than 1.5 years after the treatment for HSCT.

The second patient (patient 2) was diagnosed with HSCT-TMA on the 19 Apr. 2018 having had a HSCT transplant on the 9 Feb. 2018 for treatment of high risk acute myeloid leukaemia. At presentation the patient had lung GVHD, raised LDH, red blood cell fragments, thrombocytopenia, hemolytic anemia, proteinuria, elevated sC5b9 and was Coomb's negative. The patient received nomacopan dosing described in Table 1 starting on the 9 Jul. 2018 (more than 2.5 months after diagnosis of TMA) and resolved many of her symptoms of HSCT-TMA within 28 days of initiating nomacopan (FIG. 3), However her lung GVHD worsened and she died of complications caused by the lung GVHD shortly after coming off nomacopan.

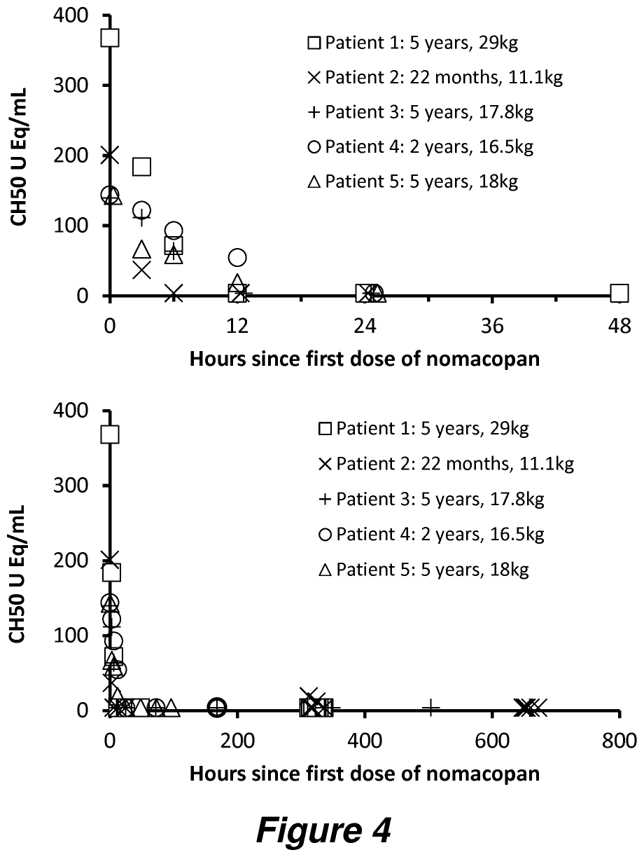

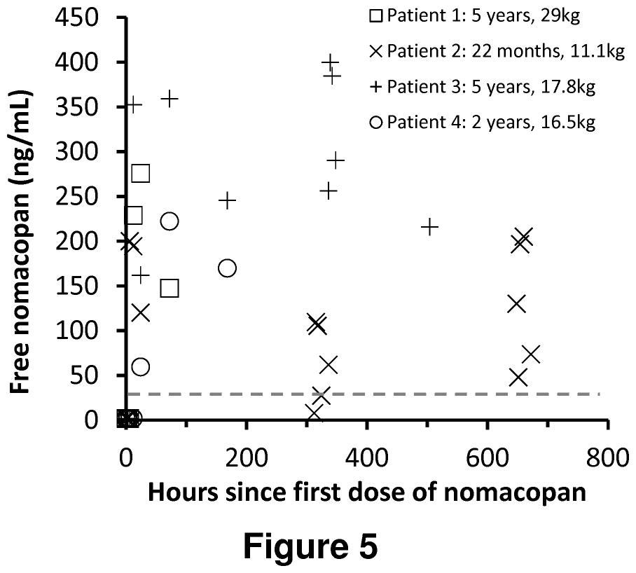

In both of these patients and three other HSCT-TMA named patients (patients 3-5) terminal complement activity measured by CH50 ELISA (FIG. 4) and free nomacopan levels were assessed (FIG. 5). The data show that the doses of nomacopan used to treat the HSCT-TMA pediatric patients rapidly and completely inhibited complement activity.

TABLE 1
dosing regimen

| Patient | Age; | Ablating | Initiation | Ablating | Initiation | Maintenance | |
|---|---|---|---|---|---|---|---|
| ID | Weight | Dose 1 | Dose 1 | Dose 2 | Dose 2 | Dose | Outcome |
| Patient | 5 years; | 38 mg | 10.4 mg | N/A | N/A | 20.7 mg | TMA |
| 1 | 23 kg | and 19 | every 12 | | | every 12 | resolved, |
| | | mg 12 | hours | | | hours from | survived to |
| | | hours | from Day | | | Day 22-56 | Day 200+ |
| | | later ^{a} | 2-21 | | | | |
| Patient | 2 years; | 18.3 mg | 5 mg | 18.3 mg | 6.7 mg | N/A | TMA |
| 2 | 12 kg | and 9.2 | every 12 | and 9.2 | every | | resolved, |
| | | mg 12 | hours | mg 12 | 12 hours | | died of lung |
| | | hours | from Day | hours | from | | GVHD |
| | | later ^{a} | 2-16 | later at | Day 18- | | |
| | | | | Day 17^{a} | EOS | | |
| Patient | 5 years; | 19.6 mg | 8 mg | N/A | N/A | N/A | Incomplete |
| 3 | 17.8 kg | and 9.8 | every 12 | | | | information |
| | | mg 12 | hours | | | | TMA |
| | | hours | from Day | | | | response, |
| | | later | 2-EOS | | | | died of |
| | | | | | | | fungal |
| | | | | | | | infection |
| Patient | 2 years; | 18.2 mg | 7.4 mg | N/A | N/A | N/A | Incomplete |
| 4 | 16.5 kg | and 9.1 | every 12 | | | | information |
| | | mg 12 | hours | | | | TMA |
| | | hours | from Day | | | | response, |
| | | later | 2-EOS | | | | died of |
| | | | | | | | infection |
| Patient | 5 years; | 18.5 mg | 6.9 mg | N/A | N/A | N/A | Incomplete |
| 5 | 18.5 kg | and 9.25 | every 12 | | | | information, |
| | | mg 12 | hours | | | | died |
| | | hours | from Day | | | | |
| | | later | 2-EOS | | | | |

EOS = end of study;
GVHD = graft versus host disease;
N/A = not applicable;
TMA = thrombotic microangiopathy.
^{a} First 2 subjects (patient 1 and patient 2) received a higher ablating dose on a mg/kg basis than patients 3-5.

### REFERENCES

[1] Hoover et al, 1984, Proc. Nat. Acad. Sci. U.S.A. 81, 2191-2193

[2] Harrison and Murphy, 1995, J. Biol. Chem. 270, 17273-17276

[3] Ford-Hutchinson, 1990, Crit. Rev. Immunol. 10, 1-12

[4] Showell et al., 1995, J. Pharm. Exp. Ther. 273, 176-184

[5] Klaas et al, 2005 J. Exp. Med. 201, 1281-1292

[6] Del Prete et al, 2007 Blood, 109, 626-631

[7] Miyahara et al, 2006 A llergol Int. 55, 91-7

[8] Taube et al, 2006 J. Immunol. 176, 3157-3164

[9] Yamaoka et al, 1989 J. Immunol. 143, 1996-2000

[10] Yokomizo et al, 1997 Nature 387, 620-624

[11] Yokomizo et al, 2000 J. Exp. Med. 192, 421-432

[12] Tager and Luster, 2003 Prostaglandins Leukot. Essent. Fatty Acids 69, 123-134

[13] Yokomizo et al., 2001, J. Biol. Chem. 276, 12454-12459

[14] Kim, N. D. and Luster, A. D. (2007) The Scientific World Journal 7, 1307-1328.

[15] Sebaldt et al., 1990 Proc Natl Acad Sd. U.S.A. 8, 6974-6978

[16] Curry et al., 2005 Journal of the American Animal Hospital Association 41 , 298-309

[17] Dube et al., 1998. Zileuton: the first leukotriene inhibitor for use in the management of chronic asthma. In: Drazen J M, Dahlen S, Lee T H, eds. Five-lipoxygenase Products in Asthma. New York, N.Y.: Marcel Dekkar, Inc

[18] Sharma and Mohammed, 2006 Immunopharmacology 14, 10-16

[19] Laskin B L, et. al, Blood 2011; 118(6): 1452-1462.

[20] George J N; Bone Marrow Transplant; 2008: 41(11) :917-91.

[21] WO2004/106369

[22] Jore, M. M. et al, Nature Structural & Molecular Biology 2016 volume 23, pages 378-386

[23] Laskin B L, et. al, Blood 2011; 118(6): 1452-1462.

[24] Cho B S, et. al. Transplantation. 2010; 90:918-26.

[25] Iacopino P, et. al. Bone Marrow Transplant 1999;24:47-51

[26] WO2016/198133

[27] Rezende et. al. J Exp Med. 2017 Nov. 6;214(11):3399-3415

[28] https://emedicine.medscape.com/article/429037-overview

[29] Jodele S, et. al. Blood. 2013; 122 (12): 2003-2007

[30] Jodele S, et. al. Biol Blood Marrow Transplant. 2016; 22(2):307-315.

[31] Goodship et. al. Blood Adv. 2017 Jul. 3;1(16):1254-1258

[32] Roversi, P et al Journal of Biological Chemistry 2013, 288(26) 18789-18802

[33] Guo, R. F. and P. A. Ward, Annu Rev Immunol, 2005, 23: p. 821-52

[34] Ricklin D & Lambris J, Nature Biotechnology, 25: 1265-1275 (2007)

[35] Nishimura, J et al., New Engl J. Med., 30;7: 632-639 (2014)

[36] Breustedt D. A., Schonfeld D. L., Skerra A. (2006) 1764(2):161-173.

[37] Terpe K, Appl Microbiol Biotechnol, 60: 523-33, 2003

[38] Schlapschy M, et al Protein Eng Des Sel. 2013 August;26(8):489-501

[39] Kuhn et al Bioconjugate Chem., 2016, 27 (10), pp 2359-2371

[40] Sambrook et al (2000)

[41] Fernandez & Hoeffler (1998)

[42] Ausubel et al. (1991)

[43] https://resources.rndsystems.com/pdfs/datasheets/kge006b.pdf

[44] Remington's Pharmaceutical Sciences; Mack Pub. Co., N.J. 1991

[45] Patel et al Ther. Deliv. (2014) 5(3), 337-365

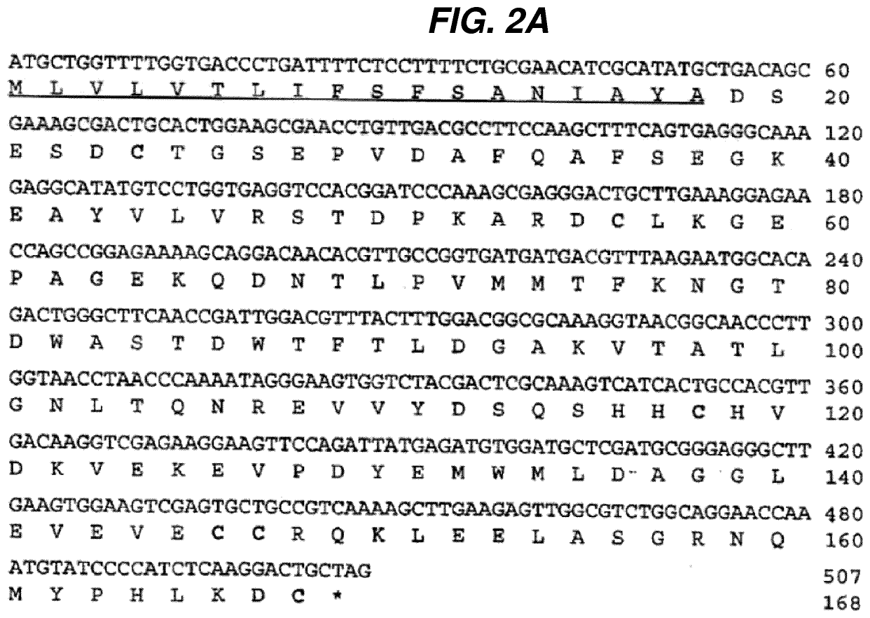

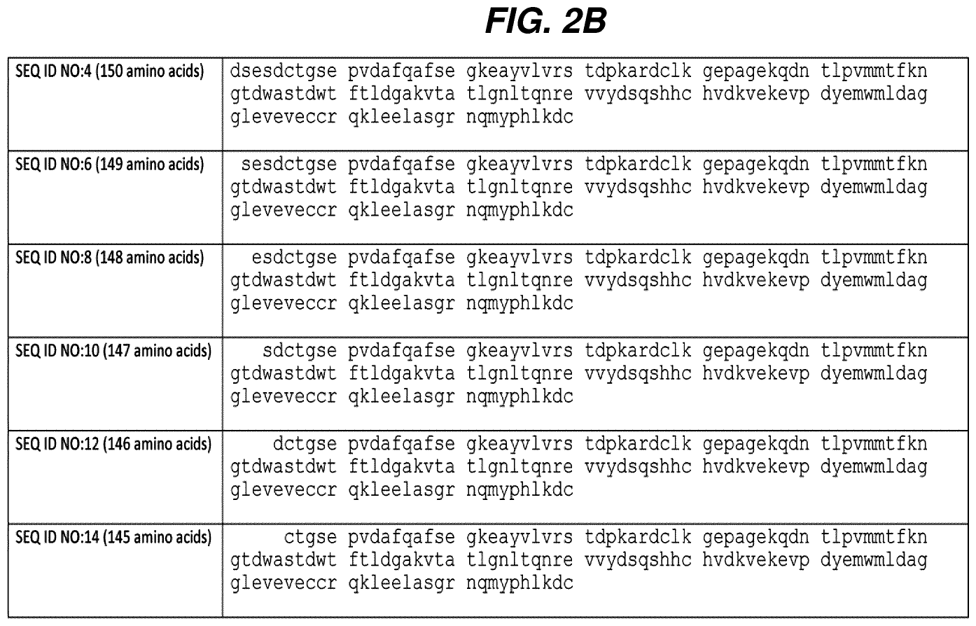

1. A method of treating or preventing a hematopoietic stem cell transplant associated thrombotic microangiopathy (HSCT-TMA) in a subject, which comprises administering to the subject a therapeutically or prophylactically effective amount of an agent which is a protein comprising amino acids 19 to 168 of the amino acid sequence in FIG. 2 (SEQ ID NO: 2) or a functional equivalent of this protein.

2. An agent which is a protein comprising amino acids 19 to 168 of the amino acid sequence in FIG. 2 (SEQ ID NO: 2) or a functional equivalent of this protein for use in a method of treating or preventing HSCT-TMA in a subject.

3. A method of treating or preventing HSCT-TMA in a subject, which comprises administering to the subject a therapeutically or prophylactically effective amount of an, agent which is a nucleic acid molecule encoding a protein comprising amino acids 19 to 168 of the amino acid sequence in FIG. 2 (SEQ ID NO: 2) or a functional equivalent of this protein.

4. An agent which is a nucleic acid molecule encoding a protein comprising amino acids 19 to 168 of the amino acid sequence in FIG. 2 (SEQ ID NO: 2) or a functional equivalent of this protein for use in a method of treating or preventing HSCT-TMA in a subject

5. The method of any one of claim 1 or 3 or the agent for use of any one of claim 2, or 4, wherein the agent is, or encodes, a protein comprising a sequence having at least 90% sequence identity to the sequence of amino acids 19 to 168 of SEQ ID NO: 2, and said protein binds C5 to prevent the cleavage of complement C5 by convertase into complement C5a and complement C5b and binds to LTB4.

6. The method of any one of claim 1, 3 or 5 or the agent for use of any one of claim 2, 4 or 5, wherein the agent is, or encodes, a protein comprising a sequence having at least 95% sequence identity to the sequence of amino acids 19 to 168 of SEQ ID NO: 2, and said protein binds C5 to prevent the cleavage of complement C5 by convertase into complement C5a and complement C5b and binds to LTB4.

7. The method of any one of claims 1, 3 or 5 to 6, or the agent for use of any one of claims 2, 4 or 5 to 6, wherein the agent is, or encodes, a protein comprising or consisting of the sequence of amino acids 19 to 168 of SEQ ID NO: 2.

8. The method of any one of claim 1 or 3, or the agent for use of any one of claim 2 or 4, wherein agent is, or encodes, a protein comprising the sequence of amino acids 19 to 168 of SEQ ID NO: 2, in which up to 50 amino acid substitutions, insertions or deletions have been made, and the protein binds C5 to prevent the cleavage of complement C5 by convertase into complement C5a and complement C5b and binds to LTB4,
wherein each of the six cysteine amino acids at positions 6, 38, 100, 128, 129, 150 of the mature nomacopan molecule as set out in SEQ ID NO: 4 is retained and at least five, ten or fifteen or each of the LTB4 binding residues and at least five, ten or fifteen or twenty or each of C5 binding residues set is retained or is subject to a conservative modification,
wherein the LTB4 binding residues are Phe18, Tyr25, Arg36, Leu39, Gly41, Pro43, Leu52, Va154, Met56, Phe58, Thr67, Trp69, Phe71, Gln87, Arg89, His99, His101, Asp103, and Trp115 (numbering according to SEQ ID NO:4) and the C5 binding residues are Va126, Va128, Arg29, Ala44, Gly45, Gly61, Thr62, Ser97, His99, His101, Met 114, Met 116, Leu117, Asp118, Ala119, Glyl20, Glyl21, Leu122, Glu123, Va1124, Glu125, Glu127, His146, Leu147 and Asp 149 (numbering according to SEQ ID NO:4).

9. The method or agent for use of claim 8 wherein up to 2, 3, 4, 5, 10, 15, 20 of the LTB4 and C5 binding residues are subject to a conservative modification.

10. The method or agent for use of claim 8 or 9 wherein at least five, ten or fifteen or each of the LTB4 binding residues and at least five, ten or fifteen or twenty or each of C5 binding residues is retained.

11. The method or agent for use of any of claims 8 to 10 wherein each of the LTB4 binding residues and each of C5 binding residues is retained or is subject to a conservative modification.

12. The method or agent for use of any of claims 8 to 11 wherein each of the LTB4 binding residues and each of C5 binding residues is retained or is subject to a conservative modification, wherein up to 2, 3, 4, 5, 10, 15, 20 of the C5 and/or LTB4 binding residues are subject to a conservative modification.

13. The method or agent for use of any of claims 8 to 12, wherein each of the LTB4 binding residues and each of C5 binding residues is retained.

14. The method of any one of claim 1 or 3, or the agent for use of any one of claim 2 or 4, wherein the agent is, or encodes, a fragment of the protein as defined in any of the preceding claims, and the protein binds C5 to prevent the cleavage of complement C5 by convertase into complement C5a and complement C5b and binds to LTB4.

15. The method or the agent for use of any preceding claim, wherein the agent is administered subcutaneously or intrasynovially, preferably subcutaneously.

16. The method or the agent for use of any preceding claim, wherein the subject is a human.

17. The method or the agent for use of any preceding claim, wherein the method further comprises treating GVHD in the subject.

18. The method or the agent for use of claim 17, wherein the GVHD is acute GVHD.

19. The method or agent for use of any preceding claim, wherein there the method comprises administering to the subject an initial ablating regimen of the agent and then administering maintenance doses of the agent, optionally wherein there is an initial maintenance dose and one or more further maintenance doses.

20. The method or the agent for use of any preceding claim, wherein the method further comprises the administration of a second HSCT-TMA treatment.

21. The method or the agent for use of claim 20, wherein the second HSCT-TMA treatment is selected from (i) a second complement inhibitor; (ii) dose reduction or complete withdrawl of calcineurin inhibitors; (iii) plasma exchange; (iv) an anti-CD20 antibody; (v) an anti-CD25 antibody; (vi) defibrotide; (vii) a vinca alkaloid, such as vincristine and (viii) a statin.

22. The method or the agent for use of claim 21 wherein:
(a) the second complement inhibitor is eculizumab or OMS721;
(b) the anti-CD20 antibody is rituximab;
(c) the anti-CD25 antibody is daclizumab;
(d) the vinca alkaloid is vincristine; and/or
(e) the statin is pravastatin.

23. The method or the agent for use of any preceding claim wherein the functional equivalent of the protein comprising amino acids 19 to 168 of SEQ ID NO:2 is a fusion protein comprising (a) a sequence as defined in any of claims 6 to 14, and (b) a second sequence and said fusion protein binds C5 to prevent the cleavage of complement C5 by convertase into complement C5a and complement C5b and binds LTB4.

24. The method or agent for use of claim 23 wherein said second sequence is a PAS sequence.

25. The method or agent for use of claim 23 or 24, wherein said fusion protein comprises multiple copies of one of ASPAAPAPASPAAPAPSAPA (SEQ ID NO: 15);
AAPASPAPAAPSAPAPAAPS (SEQ ID NO: 16); APSSPSPSAPSSPSPASPSS (SEQ ID NO: 17), SAPSSPSPSAPSSPSPASPS (SEQ ID NO: 18), SSPSAPSPSSPASPSPSSPA (SEQ ID NO: 19), AASPAAPSAPPAAASPAAPSAPPA (SEQ ID NO: 20) and ASAAAPAAASAAASAPSAAA (SEQ ID NO: 21), preferably 20-30 or 30 copies of one of SEQ ID NOs 15-21.

26. The method or agent for use of any of claims 23 to 25, wherein said fusion protein comprises (a) a PAS sequence consisting of 30 copies of SEQ ID NO:15 and (b) (i) amino acids 19-168 of SEQ ID NO:2, wherein (a) is fused to the N terminus of (b).

27. The method or agent for use of any of claims 23 to 26, wherein said fusion protein comprises the sequence of SEQ ID NO:22.

28. The method or agent for use of any one of claims 1 to 27, wherein the protein or fusion protein binds C5 to prevent the cleavage of complement C5 by convertase into complement C5a and complement C5b and binds LTB4.


SEQUENCE LISTING (23 sequences)

SEQ ID NO: 1 - DNA, 507 bp, Ornithodoros moubata
atgctggttttggtgaccctgattttctccttttctgcgaacatcgcatatgctgacagcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 2 - protein, 168 aa, Ornithodoros moubata
MLVLVTLIFSFSANIAYADSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 3 - DNA, 453 bp, Ornithodoros moubata
gacagcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 4 - protein, 150 aa, Ornithodoros moubata
DSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 5 - DNA, 450 bp, Ornithodoros moubata
agcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 6 - protein, 149 aa, Ornithodoros moubata
SESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 7 - DNA, 447 bp, Ornithodoros moubata
gaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 8 - protein, 148 aa, Ornithodoros moubata
ESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 9 - DNA, 444 bp, Ornithodoros moubata
agcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 10 - protein, 147 aa, Ornithodoros moubata
SDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 11 - DNA, 441 bp, Ornithodoros moubata
gactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 12 - protein, 146 aa, Ornithodoros moubata
DCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 13 - DNA, 438 bp, Ornithodoros moubata
tgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 14 - protein, 145 aa, Ornithodoros moubata
CTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 15 - protein, 20 aa, Artificial Sequence
  PAS sequence
ASPAAPAPASPAAPAPSAPA

SEQ ID NO: 16 - protein, 20 aa, Artificial Sequence
  PAS sequence
AAPASPAPAAPSAPAPAAPS

SEQ ID NO: 17 - protein, 20 aa, Artificial Sequence
  PAS sequence
APSSPSPSAPSSPSPASPSS

SEQ ID NO: 18 - protein, 20 aa, Artificial Sequence
  PAS sequence
SAPSSPSPSAPSSPSPASPS

SEQ ID NO: 19 - protein, 20 aa, Artificial Sequence
  PAS sequence
SSPSAPSPSSPASPSPSSPA

SEQ ID NO: 20 - protein, 24 aa, Artificial Sequence
  PAS sequence
AASPAAPSAPPAAASPAAPSAPPA

SEQ ID NO: 21 - protein, 20 aa, Artificial Sequence
  PAS sequence
ASAAAPAAASAAASAPSAAA

SEQ ID NO: 22 - protein, 751 aa, Artificial Sequence
  PAS-nomacopan
ASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAADSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 23 - protein, 150 aa, Artificial Sequence
  Nomacopan variant 2
DSESDCTGSEPVDAFQAFSEGKEAYVLVRSTDPKARDCLKGEPNGEKQDNTLPVMMTFKNGTDWASTDWTFTLDGAKVTATLGNLTQNREVVYDSQSHHCHVDKVEKEVPDYEMWQSDAGADAVEVECCRQKLEELASGRNQMYPHLKGC